(19) United States
(12) Patent Application Publication
(10) Pub. No.: US 2006/0147962 A1
(43) Pub. Date: Jul. 6, 2006

(54) Genotype test

(75) Inventors: Paul G. Jones, Leics (GB); Neale Fretwell, Leics (GB)

Correspondence Address: FULBRIGHT & JAWORSKI, LLP, 1301 MCKINNEY, SUITE 5100, HOUSTON, TX, 77010-3095, US

(73) Assignee: Mars, Inc., McLean, VA (US)

(21) Appl. No.: 11/303,853

(22) Filed: Dec. 16, 2005

Related U.S. Application Data
(60) Provisional application No. 60/738,293, filed on Nov. 18, 2005.

(30) Foreign Application Priority Data
Jun. 14, 2004 (WO) PCT/GB04/02559
Jun. 16, 2003 (GB) GB 0313964.9

(51) Int. Cl. C12Q 1/68

(52) U.S. Cl. 435/6

(57) ABSTRACT

The present invention provides a method for assessing a nutritional requirement, disease susceptibility or behavioral characteristic of a dog, the method comprising: determining the nucleotide present at one or more SNP positions in the dog genome; identifying therefrom the genetic breed inheritance of the dog; thereby determining a nutritional requirement, disease susceptibility or behavioral characteristic of the dog.

### CROSS-REFERENCE TO RELATED APPLICATIONS

This application is a continuation-in-part of International Application No. PCT/GB04/002559 filed Jun. 16, 2004 that claims priority to United Kingdom Application No. 0313964.9 filed Jun. 16, 2003. This application also claims priority to U.S. Provisional Application No. 60/738,293 filed Nov. 18, 2005. All applications are incorporated herein in their entirety.

### TECHNICAL FIELD

The invention relates to a method for determining the nutritional, medical or behavioral needs of a dog. The invention further relates to a method of determining the breed of a dog and a method of determining how closely related dogs are within a single breed.

### BACKGROUND OF THE INVENTION

The domestic dog (Canis familiaris) species comprises a large number of distinct breeds. Hundreds of different dog breeds have been established. Popular dog breeds include Labrador retrievers, Golden retrievers, German shepherds, Dachshunds, Shih Tzu, Yorkshire terriers, Poodles, Rottweilers, Boxers and Cocker spaniels.

Dog breeds typically differ in size, conformation, behavior and physiology. Different breeds can vary in size by as much as two orders of magnitude, and have differing metabolic and nutritional requirements. Particular dog breeds also have food sensitivities or predisposition to disease, which require preventative treatments and/or diets. Differences also exist in the digestibility of nutrients among breeds.

### BRIEF SUMMARY OF THE INVENTION

The invention allows the determination of the needs and characteristics of a dog, based on detection of SNPs (single nucleotide polymorphisms) in the dog. The invention takes advantage of the breed structure of the dog population to provide a genetic test for determining the nutritional, medical and behavioral needs of a dog by detecting particular SNPs in the dog. These needs may be ones for which the underlying genetic basis is unknown. The genetic test of the invention thus allows knowledge of breed-specific characteristics to be applied to addressing the specific needs of a dog. The invention additionally provides SNP sequences that can be used in the genetic test.

Accordingly the invention provides:

a method for assessing a nutritional requirement, disease susceptibility or behavioral characteristic of a dog, the method comprising:

(a) determining the nucleotide present at one or more SNP positions in the dog's genome;

(b) identifying therefrom the genetic breed inheritance of the dog;

(c) thereby determining a nutritional requirement, disease susceptibility or behavioral characteristic of the dog;

a method of determining the genetic breed background of a dog, the method comprising:

(a) determining the nucleotide present at one or more SNP positions in the dog's genome; and

(b) identifying therefrom the genetic breed inheritance of the dog;

an isolated polynucleotide that comprises a sequence of any one of SEQ ID NO:s 1 or 4 to 23 or a polypeptide encoded by any one of SEQ ID NO:s 1 or 4 to 23;

a probe, primer or antibody which is capable of detecting a polynucleotide or polypeptide according to the present invention;

a kit for carrying out the method of the invention, comprising means for detecting the nucleotide present at one or more breed-specific SNP positions;

a method of identifying one or more SNP marker(s) which can be used to determine the breed inheritance of a dog, the method comprising:

(a) screening the nuclear genome, RNA or proteins of dogs from one or more defined breeds;

(b) identifying one or more SNP positions in the nuclear genome, RNA or proteins; and

(c) determining the relationship between the nucleotide present at one or more SNP positions and one or more dog breeds.

a method of preparing customized food for a dog, the method comprising:

(a) determining one or more nutritional requirements of the dog by a method of the invention;

(b) generating a customized dog food formulation that corresponds to the nutritional requirements of the dog; and

(c) preparing a dog food according to the customized dog food formulation;

a method of providing food customized to the nutritional requirements of a dog, the method comprising providing to:

(a) the dog's owner, the person responsible for feeding the dog or a vet; or (b) to the dog;

a food which contains components suitable for the breed(s) which have contributed to the genetic breed inheritance of the dog, and which does not contain components that are not suitable for the breed(s) which have contributed to the genetic breed inheritance of the dog, wherein the breed inheritance of the dog has been determined by detecting the presence or absence of one or more breed-specific genomic SNP marker(s) in the dog;

a labeled dog food product, wherein the food product is customized for one or more breeds and the label provides an indication of one or more breed specific genomic SNPs present in said breed(s);

a method of treating a dog for a disease that occurs in a dog breed, the method comprising administering to the dog an effective amount of a therapeutic compound which prevents or treats the disease, wherein the dog has been identified as being susceptible to that disease by a method according to the present invention;

a database comprising information relating to breed-specific genomic SNPs and optionally the nutritional, medical or behavioral needs of said breeds;

a method for determining a nutritional requirement, disease susceptibility or behavioral characteristic of a dog, the method comprising:

(i) inputting data of one or more breed-specific genomic SNP positions in the dog to a computer system;

(ii) comparing the data to a computer database, which database comprises information relating to breed-specific SNPs and the nutritional requirements, disease susceptibility or behavioral characteristics of the breeds; and

(iii) determining on the basis of the comparison a nutritional requirement, disease susceptibility or behavioral characteristic of the dog;

a method for identifying the genetic breed inheritance of a dog, the method comprising:

(i) inputting genetic data from the dog to a computer system;

(ii) comparing the data to a computer database, which database comprises information relating to breed-specific gen6mic SNPs; and

(iii) determining on the basis of the comparison the nucleotide present at one or more breed-specific SNP positions, thereby identifying the breed inheritance of the dog;

a computer program comprising program code that, when executed on a computer system, instructs the computer system to perform a method according to the invention;

a computer system arranged to perform a method according to the invention comprising:

(i) means for receiving data of the nucleotide present at one or more breed-specific genomic SNP positions in the dog;

(ii) a module for comparing the data with a database comprising information relating to breed-specific genomic SNPs and the nutritional requirements, disease susceptibility or behavioral characteristics of the breeds; and

(iii) means for determining on the basis of said comparison a nutritional requirement, disease susceptibility or behavioral characteristic of the dog;

a computer system arranged to perform a method according to the invention comprising:

(i) means for receiving genetic data from the dog;

(ii) a module for comparing the data with a database comprising information relating to breed-specific genomic SNPs; and

(iii) means for determining on the basis of said comparison the breed inheritance of the dog;

a method of determining the degree of relatedness between two dogs of the same breed, the method comprising comparing the genetic breed inheritance of a dog with the genetic breed inheritance of another dog of the same breed, and determining from the comparison the degree of relatedness between the two dogs;

a method of selecting one or more dogs for breeding with a subject dog, the method comprising:

(a) comparing the genetic breed inheritance of the subject dog with the genetic breed inheritance of each dog in a test group of two or more dogs of the same breed and of the opposite sex to the subject dog;

(b) determining from the comparison the degree of relatedness between the subject dog and each dog in the test group; and

(c) selecting one or more dogs from the test group for breeding with the subject dog;

a method of providing a recommendation of one or more dogs for breeding with a subject dog, wherein the one or more dogs are selected by a method of the invention;

a method of breeding dogs, wherein a subject dog is bred with a dog selected by a method of the invention;

a database comprising information relating to the genetic breed background and sex of one or more dogs of the same breed and optionally the breeding status, age, geographical location, medical history, disease susceptibility or a physical characteristic of said dogs;

a method of selecting one or more dogs for breeding with a subject dog, the method comprising:

(i) inputting data relating to the genetic breed inheritance of a subject dog to a computer system;

(ii) comparing the data to a computer database, which database comprises information relating to the genetic breed background and sex of each dog in a test group of two or more dogs of the same breed;

(iii) determining on the basis of the comparison the degree of relatedness between the subject dog and each dog in the test group; and

(iv) selecting one or more dogs from the test group for breeding with the subject dog;

a computer system arranged to perform a method of the invention comprising:

(i) means for receiving data of the genetic breed inheritance of a subject dog;

(ii) a module for comparing the data with a database comprising information relating to the genetic breed background and sex of each dog in a test group of two or more dogs of the same breed;

(iii) means for determining on the basis of said comparison the degree of relatedness between the subject dog and each dog in the test group; and

(iv) means for selecting one or more dogs from the test group for breeding with the subject dog.

### BRIEF DESCRIPTION OF THE DRAWINGS

FIGS. 1 and 2 illustrate schematically embodiments of functional components arranged to carry out the present invention.

### BRIEF DESCRIPTION OF THE SEQUENCE LISTING

SEQ ID NO: 1 sets out the nucleic acid sequence of the English Mastiff mast cell chymase gene containing the C5375T SNP.

SEQ ID NO: 2 sets out the sequence of the forward primer used to amplify the English Mastiff mast cell chymase gene sequence containing the C5375T SNP.

SEQ ID NO: 3 sets out the sequence of the reverse primer used to amplify the English Mastiff mast cell chymase gene sequence containing the C5375T SNP.

SEQ ID NO: 4 sets out the RAGE_{—}8Kb_{—}6000 contig nucleic acid sequence.

SEQ ID NO: 5 sets out the RAGE_{—}8Kb_{—}6002 contig nucleic acid sequence.

SEQ ID NO: 6 sets out the RAGE_{—}8Kb_{—}5959 contig nucleic acid sequence.

SEQ ID NO: 7 sets out the FCGR3B_{—}7.42Kb_{—}5238 contig nucleic acid sequence.

SEQ ID NO: 8 sets out the PAI1_{—}10Kb_{—}2979 contig nucleic acid sequence.

SEQ ID NO: 9 sets out the RAGE_{—}8Kb_{—}6006 contig nucleic acid sequence.

SEQ ID NO: 10 sets out the FCGR3B_{—}7.42Kb_{—}5264 contig nucleic acid sequence.

SEQ ID NO: 11 sets out the FCGR3B_{—}7.42Kb_{—}5137 contig nucleic acid sequence.

SEQ ID NO: 12 sets out the FCGR3B_{—}7.42Kb_{—}5002 contig nucleic acid sequence.

SEQ ID NO: 13 sets out the FCGR3B_{—}7.42Kb_{—}5167 contig nucleic acid sequence.

SEQ ID NO: 14 sets out the RAGE_{—}8Kb_{—}5820 contig nucleic acid sequence.

SEQ ID NO: 15 sets out the FCGR2A_{—}9.86Kb_{—}8708 contig nucleic acid sequence.

SEQ ID NO: 16 sets out the RAGE_{—}8Kb_{—}5847 contig nucleic acid sequence.

SEQ ID NO: 17 sets out the RAGE_{—}5Kb_{—}4329 contig nucleic acid sequence.

SEQ ID NO: 18 sets out the RAGE_{—}5Kb_{—}4766 contig nucleic acid sequence.

SEQ ID NO: 19 sets out the RAGE_{—}8Kb_{—}6182 contig nucleic acid sequence.

SEQ ID NO: 20 sets out the FCGR3B_{—}7.42Kb_{—}5239 contig nucleic acid sequence.

SEQ ID NO: 21 sets out the RAGE_{—}8Kb_{—}5771 contig nucleic acid sequence.

SEQ ID NO: 22 sets out the RAGE_{—}5Kb_{—}4805 contig nucleic acid sequence.

SEQ ID NO: 23 sets out the FCGR3B_{—}7.42Kb_{—}4947 contig nucleic acid sequence.

### DETAILED DESCRIPTION OF THE INVENTION

The present invention allows the identification of the nutritional requirements, disease susceptibility or behavioral characteristics of a dog by determination of its breed ancestry. Detection of the presence or absence of SNP markers in the dog allows identification of the breeds that have contributed to the dog's genome (i.e. its genetic breed inheritance), allowing the genetic background of the dog to be deduced.

Dog Breeds

A breed is a homogeneous group of animals within a species, which has been developed by man. Dog breeds are normally divided into seven categories, based on the uses for which the breeds were originally developed. The seven dog breed categories and examples of specific breeds that fall within each category are shown in Table 1.

TABLE 1

| | | | | | |
|---|---|---|---|---|---|
| Breed | Size | Grooming | Exercise | Locality | Life-span |

a) Hounds

| | | | | | |
|---|---|---|---|---|---|
| Afghan Hound | L | Con | Con | C | B |
| Basenji | M | Lt | Mod | T/C | B |
| Basset Bleu De Gascogne | M | Lt | Con | C | B |
| Basset Fauve De Bretagne | M | Mod | Con | T/C | B |
| Basset Griffon Vendeen (Grand) | M | Mod | Con | C | B |
| Basset Griffon Vendeen (Petit) | M | Mod | Con | T/C | B |
| Basset Hound | M | Lt | Con | T/C | B |
| Bavarian Mountain Hound | M | Lt | Con | C | B |
| Beagle | M | Lt | Con | T/C | B |
| Bloodhound | L | Lt | Con | C | A |
| Borzoi | L | Mod | Con | C | B |
| Dachshund (Long Haired) | M | Mod | Mod | T/C | B |
| Dachshund (Miniature Long Haired) | S | Mod | Mod | T/C | C |
| Dachshund (Smooth Haired) | M | Lt | Mod | T/C | B |
| Dachshund (Miniature Smooth Haired) | S | Lt | Mod | T/C | C |
| Dachshund (Wire Haired) | M | Mod | Mod | T/C | B |
| Dachshund (Miniature Wire Haired) | S | Mod | Mod | T/C | C |
| Deerhound | L | Mod | Con | C | B |
| Norwegian Elkhound | L | Mod | Con | T/C | B |
| Finnish Spitz | M | Mod | Mod | C | B |
| Foxhound | L | Lt | Con | C | B |
| Grand Bleu De Gascogne | L | Lt | Con | C | B |
| Greyhound | L | Lt | Mod | C | B |
| Hamiltonstovare | L | Lt | Con | C | B |
| Ibizan Hound | L | Lt | Con | T/C | B |
| Irish Wolfhound | XL | Mod | Con | C | A |
| Norwegian Lundehund | M | Lt | Mod | T/C | B |
| Otterhound | L | Mod | Con | C | B |
| Pharaoh Hound | L | Lt | Con | C | B |
| Rhodesian Ridgeback | L | Lt | Con | T/C | B |
| Saluki | L | Mod | Con | C | B |
| Segugio Italiano | L | Lt | Con | C | B |
| Sloughi | L | Lt | Con | C | B |
| Whippet | M | Lt | Con | T/C | B |

b) Working Dogs

| | | | | | |
|---|---|---|---|---|---|
| Alaskan Malamute | L | Con | Con | C | B |
| Beauceron | L | Lt | Con | C | B |
| Bernese Mountain Dog | XL | Mod | Mod | T/C | A |
| Bouvier Des Flandres | L | Con | Con | T/C | B |
| Boxer | L | Lt | Con | T/C | B |
| Bullmastiff | L | Lt | Con | T/C | B |
| Canadian Eskimo Dog | L | Mod | Con | C | B |
| Dobermann | L | Lt | Con | T/C | B |
| Dogue de Bordeaux | L | Lt | Mod | C | B |
| German Pinscher | M | Lt | Mod | T/C | B |
| Greenland Dog | L | Mod | Con | C | B |
| Giant Schnauzer | L | Con | Con | T/C | B |
| Great Dane | XL | Lt | Con | T/C | A |
| Hovawart | L | Mod | Con | T/C | B |
| Leonberger | XL | Mod | Con | C | B |
| Mastiff | XL | Lt | Mod | C | A |
| Neapolitan Mastiff | XL | Lt | Mod | C | A |
| Newfoundland | XL | Con | Con | C | B |
| Portuguese Water Dog | L | Con | Mod | T/C | B |
| Rottweiler | L | Lt | Con | T/C | B |
| Russian Black Terrier | L | Con | Con | T/C | B |
| St. Bernard | XL | Con | Mod | T/C | A |
| Siberian Husky | L | Mod | Con | C | B |
| Tibetan Mastiff | XL | Mod | Mod | T/C | B |

c) Terrier

| | | | | | |
|---|---|---|---|---|---|
| Airedale Terrier | L | Con | Mod | T/C | B |
| Australian Terrier | S | Mod | Mod | T/C | B |
| Bedlington Terrier | M | Mod | Mod | T/C | B |
| Border Terrier | S | Mod | Mod | T/C | B |
| Bull Terrier | M | Lt | Mod | T/C | B |
| Bull Terrier (Miniature) | M | Lt | Mod | T/C | B |
| Cairn Terrier | S | Mod | Mod | T/C | B |
| Cesky Terrier | M | Con | Mod | T/C | B |
| Dandie Dinmont Terrier | M | Mod | Mod | T/C | B |
| Fox Terrier (Smooth) | M | Lt | Mod | T/C | B |
| Fox Terrier (Wire) | M | Con | Mod | T/C | B |
| Glen of Imaal Terrier | M | Mod | Mod | T/C | B |
| Irish Terrier | M | Mod | Mod | T/C | B |
| Kerry Blue Terrier | M | Con | Mod | T/C | B |
| Lakeland Terrier | M | Con | Mod | T/C | B |
| Manchester Terrier | M | Lt | Mod | T/C | B |
| Norfolk Terrier | S | Mod | Mod | T/C | B |
| Norwich Terrier | S | Mod | Mod | T/C | B |
| Parson Russell Terrier | M | Lt | Mod | T/C | B |
| Scottish Terrier | M | Con | Mod | T/C | B |
| Sealyham Terrier | M | Con | Mod | T/C | B |
| Skye Terrier | M | Mod | Mod | T/C | B |
| Soft Coated Wheaten Terrier | M | Con | Mod | T/C | B |
| Staffordshire Bull Terrier | M | Lt | Con | T/C | B |
| Welsh Terrier | M | Con | Mod | T/C | B |
| West Highland White Terrier | S | Con | Mod | T/C | B |

d) Gundogs (Sporting Group)

| | | | | | |
|---|---|---|---|---|---|
| Bracco Italiano | L | Lt | Con | C | B |
| Brittany | M | Lt | Con | C | B |
| English Setter | L | Mod | Con | T/C | B |
| German Longhaired Pointer | L | Mod | Con | C | B |
| German Shorthaired Pointer | L | Lt | Con | C | B |
| German Wirehaired Pointer | L | Mod | Con | C | B |
| Gordon Setter | L | Mod | Con | C | B |
| Hungarian Vizsla | L | Lt | Con | C | B |
| Hungarian Wirehaired Vizsla | L | Mod | Con | C | B |
| Irish Red and White Setter | L | Mod | Con | C | B |
| Irish Setter | L | Mod | Con | T/C | B |
| Italian Spinone | L | Mod | Con | C | B |
| Kooikerhondje | M | Mod | Mod | T/C | B |
| Lagotto Romagnolo | M | Mod | Con | C | B |
| Large Munsterlander | L | Mod | Con | T/C | B |
| Nova Scotia Duck Tolling Retriever | M | Mod | Mod | T/C | B |
| Pointer | L | Lt | Con | T/C | B |
| Retriever (Chesapeake Bay) | L | Mod | Con | C | B |
| Retriever (Curly Coated) | L | Mod | Con | C | B |
| Retriever (Flat Coated) | L | Mod | Con | T/C | B |
| Retriever (Golden) | L | Mod | Con | T/C | B |
| Retriever (Labrador) | L | Lt | Con | T/C | B |
| Spaniel (American Cocker) | M | Con | Mod | T/C | B |
| Spaniel (Clumber) | L | Mod | Mod | C | B |
| Spaniel (Cocker) | M | Con | Mod | T/C | B |
| Spaniel (English Springer) | M | Mod | Con | T/C | B |
| Spaniel (Field) | M | Mod | Con | C | B |
| Spaniel (Irish Water) | M | Mod | Con | C | B |
| Spaniel (Sussex) | M | Mod | Con | C | B |
| Spaniel (Welsh Springer) | M | Mod | Con | T/C | B |
| Spanish Water Dog | M | Mod | Mod | C | B |
| Weimaraner | L | Lt | Con | T/C | B |

e) Pastoral (Herding Group)

| | | | | | |
|---|---|---|---|---|---|
| Anatolian Shepherd Dog | L | Mod | Con | C | B |
| Australian Cattle Dog | M | Lt | Mod | C | B |
| Australian Shepherd | L | Mod | Con | C | B |
| Bearded Collie | L | Con | Mod | T/C | B |
| Belgian Shepherd Dog (Groenendael) | L | Mod | Con | T/C | B |
| Belgian Shepherd Dog (Malinois) | L | Mod | Con | T/C | B |
| Belgian Shepherd Dog (Laekenois) | L | Mod | Con | T/C | B |
| Belgian Shepherd Dog (Tervueren) | L | Mod | Con | T/C | B |
| Bergamasco | L | Con | Mod | C | B |
| Border Collie | M | Mod | Con | C | B |
| Briard | L | Con | Con | T/C | B |
| Collie (Rough) | L | Con | Con | T/C | B |
| Collie (Smooth) | L | Lt | Con | T/C | B |
| Estrela Mountain Dog | XL | Mod | Mod | C | B |
| Finnish Lapphund | M | Con | Mod | T/C | B |
| German Shepherd Dog (Alsatian) | L | Mod | Con | T/C | B |
| Hungarian Kuvasz | L | Mod | Mod | C | B |
| Hungarian Puli | M | Con | Mod | T/C | B |
| Komondor | L | Con | Mod | C | A |
| Lancashire Heeler | S | Lt | Mod | T/C | B |
| Maremma Sheepdog | L | Mod | Con | C | B |
| Norwegian Buhund | M | Mod | Mod | T/C | B |
| Old English Sheepdog | L | Con | Con | T/C | B |
| Polish Lowland Sheepdog | M | Con | Con | T/C | B |
| Pyrenean Mountain Dog | XL | Con | Mod | T/C | A |
| Pyrenean Sheepdog | M | Mod | Mod | T/C | B |
| Samoyed | L | Con | Con | T/C | B |
| Shetland Sheepdog | M | Con | Mod | T/C | B |
| Swedish Lapphund | M | Con | Mod | T/C | B |
| Swedish Vallhund | M | Lt | Mod | T/C | B |
| Welsh Corgi (Cardigan) | M | Lt | Mod | T/C | B |
| Welsh Corgi (Pembroke) | M | Lt | Mod | T/C | B |

f) Utility Dogs (Non-sporting)

| | | | | | |
|---|---|---|---|---|---|
| Akita | L | Mod | Con | T/C | B |
| Boston Terrier | S | Lt | Mod | T/C | B |
| Bulldog | M | Lt | Mod | T/C | A |
| Canaan Dog | L | Lt | Mod | T/C | B |
| Chow Chow | L | Con | Mod | T/C | B |
| Dalmatian | L | Lt | Con | T/C | B |
| French Bulldog | S | Lt | Mod | T/C | B |
| German Spitz (Klein) | S | Con | Lt | T/C | B |
| German Spitz (Mittel) | M | Con | Lt | T/C | B |
| Japanese Shiba Inu | M | Mod | Mod | T/C | B |
| Japanese Spitz | M | Con | Mod | T/C | B |
| Keeshond | M | Con | Mod | T/C | B |
| Lhasa Apso | S | Con | LT | T/C | B |
| Mexican Hairless | M | Lt | Mod | T/C | B |
| Miniature Schnauzer | S | Con | Mod | T/C | B |
| Poodle (Miniature) | M | Con | Mod | T/C | C |
| Poodle (Standard) | L | Con | Con | T/C | C |
| Poodle (Toy) | S | Con | Mod | T/C | C |
| Schipperke | S | Lt | Lt | T/C | C |
| Schnauzer | M | Con | Mod | T/C | B |
| Shar Pei | M | Lt | Mod | T/C | B |
| Shih Tzu | S | Con | Mod | T/C | B |
| Tibetan Spaniel | S | Mod | Mod | T/C | C |
| Tibetan Terrier | M | Con | Mod | T/C | B |

g) Toy Dogs

| | | | | | |
|---|---|---|---|---|---|
| Affenpinscher | S | Mod | Lt | T/C | B |
| Australian Silky Terrier | S | Mod | Lt | T/C | B |
| Bichon Frise | S | Con | Lt | T/C | B |
| Bolognese | S | Con | Lt | T/C | B |
| Cavalier King Charles Spaniel | S | Mod | Mod | T/C | B |
| Chihuahua (Long Coat) | S | Mod | Lt | T/C | B |
| Chihuahua (Smooth Coat) | S | Lt | Lt | T/C | B |
| Chinese Crested | S | Lt | Lt | T/C | B |
| Coton De Tulear | S | Con | Lt | T/C | B |
| English Toy Terrier (Black and Tan) | S | Lt | Lt | T/C | B |
| Griffon Bruxellios | S | Mod | Lt | T/C | B |
| Havanese | S | Con | Lt | T/C | B |
| Italian Greyhound | S | Lt | Mod | T/C | B |
| Japanese Chin | S | Mod | Lt | T/C | B |
| King Charles Spaniel | S | Mod | Lt | T/C | B |
| Lowchen (Little Lion Dog) | S | Con | Lt | T/C | B |
| Maltese | S | Con | Lt | T/C | B |
| Miniature Pinscher | S | Lt | Lt | T/C | B |
| Papillon | S | Mod | Lt | T/C | B |
| Pekingese | S | Con | Lt | T/C | B |
| Pomeranian | S | Con | Lt | T/C | B |
| Pug | S | Lt | Lt | T/C | B |
| Yorkshire Terrier | S | Con | Lt | T/C | B |

KEY

| | | | | |
|---|---|---|---|---|
| SIZE | S-Small | M-Medium | L-Large | XL-Ex Large |
| GROOMING | Lt-Little | Mod-Moderate | Con-Considerable | |
| EXERCISE | Lt-Little | Mod-Moderate | Con-Considerable | |
| LOCALITY | T-Town | C-Country | | |
| LIFESPAN | A-Under 9 Yrs | B-Over 9 Yrs | C-Over 15 Yrs | |

Breed-Specific SNPs

As used herein, a “breed-specific SNP” is a single nucleotide polymorphism that can be used to distinguish between different dog breeds or to determine breed inheritance, either alone or in combination with other SNPs. Such a breed-specific SNP may be unique to one breed. Alternatively, a breed-specific SNP may be present in a plurality of breeds, but its presence in combination with one or more other breed-specific SNPs can be used to determine a dog's genetic breed inheritance. In one embodiment of the invention, the SNP is present in substantially all dogs of one breed, and is absent in substantially all dogs of other breeds. The breed-specificity of a SNP is typically assessed in a sample population of a breed that is representative of that breed. Such a sample population will typically consist only of purebred dogs. The sample population typically comprises 4 or more dogs per breed, such as at least 20, 100, 400, 1000 or 10,000 dogs of one breed. For example, the sample population tested for the SNP may be up to 10, 200, 500, 1000, 10,000 or 1,000,000 or more dogs. The sample population may consist of from 4 to 10,000, for example 20 to 1000, or 100 to 500 dogs per breed. For example, the sample population may be from 200 to 400 dogs per breed.

A breed-specific SNP is typically present in 70%, 80% or 90% or more of the sample population of that breed, preferably 95% or more of the sample population, more preferably 99% or more of the sample population. The breed-specific SNP is typically absent in substantially all dogs of sample populations of other breeds. For example, a breed specific SNP may be present in 30%, 20% or 10% or less of a sample population of another breed, preferably 5% or less of the sample population, more preferably 1% or less of the sample population. In a preferred embodiment, the SNP is present in at least 95% of dogs in a sample population of from 400 to 1000 dogs of a breed and/or is present in 5% or less dogs in a sample population of from 400 to 1000 dogs of any other breed. In a most preferred embodiment, the breed-specific SNP will be unique to that breed, i.e. it will be present in 100% of dogs in a sample population which is representative of that breed and will be entirely absent from dogs in a sample population which is representative of any other breed.

Alternatively, the SNP may be specific for a breed category shown in Table 1. For example, the SNP marker may be specific for Hound breeds such as the Beagle, Bloodhound, Whippet or Greyhound. The SNP marker may be specific for Working dogs, such as the Boxer, Great Dane and St Bernard. The SNP marker may be specific for dogs in the Terrier group, such as the West Highland White Terrier and the Airedale Terrier. The SNP marker may be specific for breeds in the Utility, or Non-Sporting, group such as the Bulldog, Dalmatian and Poodle. The SNP marker may be specific for Toy dog breeds such as the Chihuahua and Shih Tzu.

In one embodiment of the invention, the SNP is specific to a family or sub-group of breeds within a breed category. The SNP may be specific for Gundogs, or Sporting group dogs. This category is divided into four sub-groups: Retriever, Spaniels, Hunt/Point/Retrieve and Setters. The SNP marker may be specific for any one or more of these four sub-groups. The SNP marker may be specific for dogs in the Pastoral, or Herding, group. This breed category includes the Collie family of breeds and Shepherd dogs. Hence the SNP may be specific for the Collie family and/or Shepherd dogs.

In a further embodiment of the invention, the breed-specific SNP can be used to distinguish one breed of dog in a panel of dog breeds from the other breeds in the panel. The panel may consist of from 2 to 400 breeds, for example from 2 to 200, from 5 to 100, from 5 to 30, from 5 to 20, from 10 to 15, from 2 to 10 or from 5 to 10 breeds. The SNP marker is thus specific for one of the breeds in the panel. The SNP marker may actually be found in more than one breed, for example for 2, 3, 5, 10 or more breeds. However, according to this particular embodiment, it will be specific for only one of the breeds in the panel. The breeds can be selected from any of the categories shown in Table 1 above. A SNP that is specific for two or more breeds within a breed category can be used to distinguish those particular breeds from other breeds in the breed category. In a most preferred embodiment, the SNP marker is present only in one breed (i.e. it is unique to that breed compared to all other breeds).

In another preferred embodiment of the invention, each breed is not defined by a single SNP, but by the combination of SNPs present in the dog genome. Accordingly, the genetic breed inheritance of a dog may be identified from a combination of the nucleotides present at two or more SNP positions, for example at three or more, four or more, five or more, or six or more SNP positions. Each dog breed may therefore be defined by a set of rules based on the combination of nucleotides found at each of these SNP positions. In some cases, in order to define a breed it may be necessary to provide one or more rules which specify the nucleotide found at least 7, 8, 9, 10, 11, 12, 15 or 20 or more SNP positions. Typically, the number of SNP positions used in each rule will be from 2 to 20, preferably from 2 to 12, more preferably from 2 to 6. Each dog breed may be defined by a single rule or more than one rule, for example by 2, 3, 4, 5, 10, 20 or more rules.

In order to identify the genetic breed inheritance of the dog, typically at least 2 different SNP positions are typed, for example at least 3, 4, 5, 6, 7, 8, 9 or 10 or more positions, preferably at least 20 different SNP positions. Typically up to 10, 15, 20, 25, 30, 50 or 100 positions will be typed, for example 10 to 50, or 10 to 25 positions. In this case, the term “typed” typically comprises determining the nucleotide present at any given SNP position.

The term “genetic breed inheritance” is used herein to describe the breed ancestry of a dog, namely the one or more breeds that have contributed to the dog's genome. Therefore, in the case of a purebred dog, the term “genetic breed inheritance” will typically correspond to the breed of the dog. Accordingly, in one embodiment of the invention the nucleotide present at one or SNP positions in the dog's genome can be used to determine the breed of the dog. In the case of a crossbred or outbred dog, the term “genetic breed inheritance” may relate to the one or more breeds that are represented in the dog's lineage. This term may further be used to describe the proportions or relative amounts of each breed that goes to make up a mongrel dog.

The method of the invention can be used to detect a genetic breed inheritance from any number of different dog breeds, such as at least 2, 3, 4, 5, 10, 20, 50, 70, 100 or 400 or more different dog breeds. In one embodiment of the invention, the nucleotide present at one or more SNP positions is used to distinguish between the following breeds: Labrador retriever, Golden retriever, German Shepherd, Dachshund, Shih Tzu, Yorkshire terrier, Poodle, Rottweiler, Boxer and Cocker spaniel.

Breed Differences

The present invention enables the determination of a nutritional requirement, disease susceptibility or behavioral characteristic of a dog, the method comprising determining the nucleotide present at one or more breed-specific SNP (single nucleotide polymorphism) positions in the dog genome and thereby determining the breed inheritance of the dog. A method for determining the breed inheritance of a dog according to the invention may be carried out by electronic means, for example by using a computer system. The presence of a breed-specific SNP in a dog indicates that it has a genetic inheritance in common with that breed, and therefore is likely to share that breed's characteristics regarding nutritional requirements, disease susceptibility and behavioral characteristics. The absence of a particular breed-specific SNP indicates that the dog does not have any genetic inheritance from that breed. In one embodiment, a method for determining the nutritional requirements, disease susceptibility or behavioral characteristics of a dog according to the invention may be carried out by electronic means, for example by using a computer system.

Dog breeds differ from each other in (for example) size, weight, shape, digestive transit time, growth period, temperament, activity level, life span, coat type, nutritional requirements and disease susceptibility. Table 1 illustrates some of these differences. The nutritional requirement, disease susceptibility or behavioral characteristic assessed by the method of the invention may be any such nutritional, medical or behavioral need mentioned herein, such as those in Table 1 or those discussed below.

Bodyweight size in dog breeds can be grouped into 5 categories (from smallest to largest): toy, small, medium, large and giant (or extra large). Breeds also differ in the ratio of gastrointestinal weight: total bodyweight. In small breeds, the digestive tract represents 7% of their total bodyweight, whilst for giant breeds this is only 2.7%. Digestive transit time also varies depending on the size of the dog, and can vary from 15 hours to 4 days. The growth period of a dog varies by breed, is determined by feeding regime and feeding rate, and lasts between approximately 8 months for a small breed to up to 24 months for a giant breed. Small breeds have a much greater growth rate than large breeds. Small breed puppies typically multiply their birth weight by approximately 20 times during their first year of life. This ratio can be as great as 100 times for giant breeds. The size of a dog also affects its life expectancy. The larger and heavier the dog, the earlier the aging process begins. Life expectancy for giant breeds is generally half that of small breeds.

Breeds differ in their dietary needs due to differences in nutrient requirement and physical form. For example, daily energy requirements to maintain body weight are higher for large, active dogs than small or inactive dogs. However, per unit of bodyweight, a small breed's energy requirements are more than twice those of large breeds. Some breeds, such as Labrador Retrievers, Basset Hounds, Beagles and Cocker Spaniels, are predisposed to obesity. Ingredient tolerance, food allergies and nutrient metabolism also differ among breeds. For example, Irish Setters often exhibit gluten intolerance. Other, well-recognized problems include vitamin A responsive dermatitis in Cocker Spaniels and zinc-responsive dermatitis in Siberian Huskies and Alaskan Malamutes. Some Cocker Spaniels and Golden Retrievers have low blood taurine levels which are responsive to dietary taruine supplements.

Breeds also differ in their susceptibility to disease. For example, Dalmatians have predisposition to deafness and to the presence of uric acid crystals in the urine. Poodles and Bichon Frise have a predisposition to periodontal disease. Bedlington Terriers and West Highland Terriers are prone to copper storage disease. German Shepherds and Beagles often experience diarrhea caused by a gastrointestinal immune deficiency. Hip dysplasia is common in a number of breeds, particular in the Herding, Working and Sporting groups. Boxers, Doberman Pinschers and Great Danes can develop dilated cardiomyopathy. Skin and hair coat problems are frequent in breeds such as the Miniature Poodle and Chow Chow. Silky Terriers and Yorkshire Terriers are susceptible to diabetes.

Behavioral differences are also marked between dog breeds. For example, the Labrador Retriever is playful, loving to people and hardworking and is suitable for jobs such as a guide dog for the disabled, a search-and-rescue dog, and for narcotics detection. Boxers are playful and fun-loving dogs, but are also strong and defensive, so early obedience training is important. Rottweilers enjoy exercise and outdoor sports, but due to a more aggressive nature may not be suitable as a pet in households with young children. Hound breeds require a significant amount of exercise, whereas Toy dog breeds such as the Chihuahua and Shih Tzu do not need a large amount of exercise. Gundogs, or Sporting group dogs, are active dogs and require plenty of attention and regular, strenuous exercise. The temperament of these breeds makes them ideal family dogs. Knowledge of breed characteristics is therefore important when selecting a dog breed for a particular job or as a pet.

Crossbred and Outbred (Mongrel) Dogs

In one embodiment of the method of the invention, the dog that is tested may be a crossbred or outbred (mongrel) dog. A crossbred dog is the offspring of two different purebred dogs. An outbred or mongrel dog is a dog of unknown parentage, or is the result of the combination of three or more different breeds. An outbred dog may therefore represent a mixture of 3 or more breeds, for example, 4, 5 or more different breeds. The breeds that contribute to an outbred dog's genetic breed inheritance may be from within the same category of breed or from different breed categories.

A mongrel will typically display a combination of physical characteristics that are not found within one particular breed, such as any characteristics mentioned herein, for example size, shape, color, coat type, stature, gait, height or head shape. For example, an outbred dog may have the size and shape of one breed, but have the color or coat type of a different breed. Therefore, the method of the invention may be used to identify the genetic breed inheritance of a dog which has a mixture of characteristics typically found in different breeds. In particular, the method may be used to determine the genetic background of a dog which has the physical features of a mongrel, or is suspected of being a mongrel. The method of the invention may also be used to identify or to confirm the genetic background of a crossbred dog.

Nutritional Requirements

The present invention provides a means of determining a nutritional requirement of a dog, based on its genetic breed inheritance. Such a nutritional requirement is any such requirement mentioned herein, for example as discussed below. The requirement typically relates to the proportions, total amounts or types of vitamins, minerals, fat, carbohydrates, fiber, protein and water required. In one aspect, the requirement may relate to whether or not the dog requires or needs to avoid particular food components.

The protein requirement of a dog may relate to the total amount of protein or type of protein needed, as defined by the protein source or amino acid composition. For example, the essential amino acids for dogs include lysine, arginine, histidine, isoleucine, leucine, methionine, cysteine, phenylalanine, tyrosine, threonine, tryptophan and valine. Essential amino acids cannot be synthesized by the dog, and so must be present in its food. The amount of each amino acid required may vary. In particular, the dog may have a requirement for an amino acid such as taurine. The dog's nutritional requirements may concern the source of protein, for example, whether the protein is derived from meat, poultry, dairy, vegetable or other protein sources. These protein sources may be defined as high or low quality protein sources. In this respect, quality is defined by digestibility and amino acid content. For example, a high quality protein source (such as animal protein) may contain all the essential amino acids and/or have high digestibility, whereas a low quality protein source (such as vegetable protein) may be missing one or more essential amino acids and/or have low digestibility.

The dog's nutritional requirement may further relate to the amount of fat needed by the dog or to a particular type of fat that is required. Fats are typically saturated, polyunsaturated or monounsaturated. A dog may require different amounts of each type of fat, or only one or more of these types of fat. For example, the nutritional requirement may be for polyunsaturated or monounsaturated fats only. Fats also differ in their fatty acid composition. The nutritional requirement may relate to particular fatty acids, such as essential fatty acids, which cannot be made within the dog's body and so have to be provided in the diet. The essential fatty acids may be classified as omega-6 and omega-3 fatty acids, such as linoleic, linolenic and arachidonic acids, for example, gamma linolenic acid (GLA). These polyunsaturated fatty acids vary in the number of carbon atoms and the degree of unsaturation, and may be classified as short-chain and long-chain fatty acids. In one aspect of the invention, the nutritional requirement may relate to the absolute amounts of these fatty acids, or to the ratio of omega-6 to omega-3 fatty acids.

In another aspect of the invention, the dog's nutritional requirement may relate to the total amounts or proportions of vitamins or minerals needed. Vitamins may be divided into two main categories: fat soluble and water soluble. The fat soluble vitamins include vitamins A, D, E and K. The water soluble vitamins include vitamins B1, B2, B6, B12, biotin, choline, pantothenic acid, nicotinic acid and folic acid. A dog may require particular levels of each vitamin. Minerals can be divided into two groups: macro-minerals and trace minerals. Macro-minerals include calcium, phosphorus, magnesium, sodium, potassium and chloride. Trace minerals include iron, zinc, copper, manganese, cobalt, selenium and iodine. The nutritional requirement of the dog may relate to the total amounts of each mineral or to the ratio between the minerals needed. For example, the nutritional requirement may relate to the ratio between calcium and phosphorus.

The dog's nutritional requirement may relate to the amount of carbohydrate or to the type of carbohydrate needed. Carbohydrates can be classified according to the glycemic index, which measures the ability of a food to elevate blood glucose levels. Carbohydrates with a high glycemic index enter the bloodstream quickly, whereas those with a low glycemic index enter the bloodstream slowly and provide sustained, longer-term energy. The glycemic index of a food is typically given in relation to glucose (or maltose), which has a nominal value of 100. For example, barley has a lower glycemic index than other grains such as corn, wheat or rice. Therefore, in one aspect of the invention, the dog's nutritional requirements may relate to the glycemic index of carbohydrate that is required. The nutritional requirement may further relate to the amount or proportion of fiber or the type of fiber needed. For example, fiber may be derived from different sources, such as fruit, vegetable or grains. Different types of fiber may differ in how quickly they are fermented. For example, fruit and vegetable fibers are moderately fermented whereas grain fibers are more slowly fermented.

The nutritional requirement of the dog may concern its metabolic or energy requirements. The energy requirement of the dog typically relates to its size and activity level. A large dog generally requires a greater total amount of energy than a small dog, and an active dog will normally require more energy than an inactive dog. For example, a dog may have low activity (<1 hour per day), moderate activity (1-2 hours per day), moderate to high activity (2-3 hours per day) or high activity (>3 hours per day). The energy requirement is usually expressed as either kilocalories (kcal) or kilojoules (kJ).

The nutritional requirement may be determined on a daily, weekly basis or a monthly basis. Preferably, the dog's nutritional requirements will be determined on a daily basis, for example as a recommended daily amount (RDA) of a nutrient. The energy requirement of the dog will typically be determined on a daily basis.

The nutritional requirement of the dog may relate to food allergies or intolerance. Allergens for dogs typically fall into one of four groups: (i) milk, eggs, soy, wheat (gluten), peanuts, shellfish, fruits, tree nuts; (ii) sesame seeds, sunflower seeds, cottonseed, poppy seed, beans, peas, lentils; (iii) tartrazine, sulphites and latex; and (iv) salicylate, amines and glutamate. The most common food allergies are to those foods in group (i).

Disease Susceptibility

The present invention allows for determination of a disease susceptibility of a dog, based on its genetic breed inheritance. Various dog breeds have susceptibility to different diseases and conditions. Such diseases or conditions may be cardiovascular, inflammatory, immunological, infectious, metabolic, endocrine or gastrointestinal in nature. The disease or condition may be any of the diseases or conditions mentioned herein. For example, German Shepherd dogs commonly suffer from hip dysplasia, epilepsy, gastric torsion (bloat), perianal fistulas and exocrine pancreatic deficiency. Labrador Retrievers are particularly susceptible to hip, elbow and retinal dysplasia, obesity and exercise-induced collapse. Golden Retrievers are also prone to hip dysplasia, and sometimes experience skin and coat problems such as pyotraumatic dermatitis (hotspots).

Dachshunds are susceptible to spinal disc injuries, diabetes, urinary stones, eye disorders, skin conditions and heart disease. Cocker Spaniels commonly suffer from hereditary eye problems (such as PRA, cataracts, glaucoma, eyelid, eyelash and retinal abnormalities), skin conditions, hemophilia, ear infections (such as otitis externa), heart disease and epilepsy. Boxers are prone to tumors, digestive problems, heart disease, corneal ulcers, skin fold infections and bloat. Rottweilers are susceptible to hip and elbow dysplasia, osteochonsrosis dessicans, panosteitis, entropieon (inverted eyelids), hypothyroidism, von Willebrand's disease and bloat. Shih Tzu dogs commonly suffer from slipped stifle (a joint disorder) and renal dysplasia. Poodles are prone to hip dyslplasia, PRA, cataracts, epilepsy, bloat, von Willebrand's disease, skin disorders and autoimmune disorders.

Behavioral Characteristics

In another aspect of the invention, a behavioral characteristic of a dog may be determined. As discussed herein, different dog breeds have different activity levels and temperament. Accordingly, dogs differ in the type of environment that is suitable for them. For example, dogs have differing requirements for space, locality (e.g. town or countryside), exercise, grooming and attention. Dog breeds also differ in their trainability, people fear, aggressiveness, alertness and cognitive performance. When selecting an appropriate environment for a dog, it is important to bear in mind factors such as their size at maturity and their temperament (e.g. aggressiveness). The behavioral characteristics determined according to the present invention may be any of those in Table 1 or any other characteristics discussed herein.

Symptoms of a Problem

In one aspect of the invention, a nutritional requirement, disease susceptibility or behavioral characteristic is determined of a dog that is suspected of having a nutritional, medical or behavioral problem. The dog may be displaying physical or psychological symptoms that are indicative of a nutritional imbalance or deficiency, a disease or behavioral problem. A nutritional or medical problem may be indicated by changes in eye color, gum and mouth tissue, skin condition, coat condition, energy level or muscle tone in the dog. Other symptoms of a problem may include lethargy, weight loss, bladder control loss, change in water intake, change in feces quality, appetite loss, sudden behavioral change or alertness change.

Detection of SNP Markers

The detection of SNPs according to the invention may comprise contacting a polynucleotide or protein of the animal with a specific binding agent for a breed-specific SNP and determining whether the agent binds to the polynucleotide or protein.

The method is typically it is carried out in vitro on a sample from the dog. The sample typically comprises a body fluid and/or cells of the individual and may, for example, be obtained using a swab, such as a mouth swab. The sample may be a blood, urine, saliva, skin, cheek cell or hair root sample. The sample is typically processed before the method is carried out, for example DNA extraction may be carried out. The polynucleotide or protein in the sample may be cleaved either physically or chemically, for example using a suitable enzyme. In one embodiment the part of polynucleotide in the sample is copied or amplified, for example by cloning or using a PCR based method prior to detecting the SNP marker(s).

Tables 2 and 7 show breed-specific SNPs that can be used to type the breed inheritance of a dog. A breed-specific SNP may be a “silent” polymorphism. Such “silent” polymorphisms are those which do not result in a change in amino acid sequence. Only SNPs that change the coding sequence of the nucleic acid sequence may be detected in polypeptide sequences. The polymorphism preferably does not affect the function of the protein in any other way, for example by altering gene expression by changing promoter activity, mRNA stability, mRNA splicing or epigenetic status. Such polymorphisms may or may not be causative of a breed phenotype. Preferably, the breed-specific SNP is not causative of a nutritional requirement, disease susceptibility or behavioral characteristic of a dog, and is not in linkage disequilibrium with such a SNP. The SNP may however be specific for one or more physical characteristics of a breed, for example size, shape, color, coat type, stature, gait, height or head shape, or other breed traits or phenotypes.

In the present invention, any one or more methods may comprise determining the nucleotide present at one or more breed-specific SNP positions in the dog. In a preferred embodiment of the invention, the nucleotide present at more than one breed-specific SNP positions is detected, such as at least 2, 3, 5, 10, 15 or 20 or more SNP positions. Preferably 10 or more breed-specific SNP positions are typed, more preferably 20 or more breed-specific positions. Any possible combination of breed-specific SNPs may be tested. In a preferred embodiment, the one or more SNP positions are any of those identified in SEQ ID NO:s 1 or 4 to 23.

The markers which are tested may be specific to a combination of different breeds. In one embodiment, the dog is tested for the presence and/or absence of one or more breed-specific SNP markers for at least 2, 3, 5 or 10 different breeds. In one embodiment the markers that are typed are specific for the following breeds: Labrador retriever, Golden retriever, German Shepherd, Dachshund, Shih Tzu, Yorkshire terrier, Poodle, Rottweiler, Boxer and Cocker spaniel. As discussed herein, in aspect of the invention the breed-specific SNP is present in substantially all dogs of that breed, and is absent in substantially all dogs of other breeds. One or more markers specific for each breed may be typed, for example at least 2, 3, 5 or 10 markers may be tested which are specific for one breed.

The breed-specific SNP is typically detected by directly determining the presence of the polymorphic sequence in a polynucleotide or protein of the dog. Such a polynucleotide is typically genomic DNA, mRNA or cDNA. The SNP may be detected by any suitable method such as those mentioned below.

A specific binding agent is an agent that binds with preferential or high affinity to the polynucleotide or polypeptide having a particular nucleotide or amino acid at a SNP position but does not bind or binds with only low affinity to polynucleotides or proteins which have a different nucleotide or amino acid at the same SNP position. The specific binding agent may be a probe or primer. The probe may be a protein (such as an antibody) or an oligonucleotide. The probes or primers will typically also bind to flanking nucleotides and amino acids on one or both sides of the SNP position, for example at least 2, 5, 10, 15 or more flanking nucleotide or amino acids in total or on each side. Thus a probe or primer may be fully or partially complementary to (i.e. have homology with) either all or part of the flanking 5′ and/or 3′ sequences shown in Tables 2 and 7. The probe may be labeled or may be capable of being labeled indirectly. The binding of the probe to the polynucleotide or protein may be used to immobilize either the probe or the polynucleotide or protein.

Generally in the method, determination of the binding of the agent to the breed-specific SNP can be done by determining the binding of the agent to the polynucleotide or protein of the dog. However in one embodiment the agent is also able to bind the corresponding wild-type sequence, for example by binding the nucleotides or amino acids which flank the SNP marker position, although the manner of binding to the wild-type sequence will be detectably different to the binding of a polynucleotide or protein containing the SNP marker.

The method may be based on an oligonucleotide ligation assay in which two oligonucleotide probes are used. These probes bind to adjacent areas on the polynucleotide which contains the SNP marker, allowing after binding the two probes to be ligated together by an appropriate ligase enzyme. However the presence of single mismatch within one of the probes may disrupt binding and ligation. Thus ligated probes will only occur with a polynucleotide that contains the SNP marker, and therefore the detection of the ligated product may be used to determine the presence of the SNP marker.

In one embodiment the probe is used in a heteroduplex analysis based system. In such a system when the probe is bound to polynucleotide sequence containing the SNP marker it forms a heteroduplex at the site where the SNP marker occurs and hence does not form a double strand structure. Such a heteroduplex structure can be detected by the use of single or double strand specific enzyme. Typically the probe is an RNA probe, the heteroduplex region is cleaved using RNAase H and the SNP marker is detected by detecting the cleavage products.

The method may be based on fluorescent chemical cleavage mismatch analysis which is described for example in PCR Methods and Applications 3, 268-71 (1994) and Proc. Natl. Acad. Sci. 85, 4397-4401 (1998).

In one embodiment a PCR primer is used that primes a PCR reaction only if it binds a polynucleotide containing the SNP marker, for example a sequence- or allele-specific PCR system, and the presence of the SNP marker may be determined by the detecting the PCR product. Preferably the region of the primer which is complementary to the SNP marker is at or near the 3′ end of the primer. The presence of the SNP marker may be determined using a fluorescent dye and quenching agent-based PCR assay such as the Taqman PCR detection system.

The specific binding agent may be capable of specifically binding the amino acid sequence encoded by a polymorphic sequence, preferably one of the sequences shown in Table 2. For example, the agent may be an antibody or antibody fragment. The detection method may be based on an ELISA system.

The method may be an RFLP based system. This can be used if the presence of the SNP marker in the polynucleotide creates or destroys a restriction site that is recognized by a restriction enzyme.

The presence of the SNP marker may be determined based on the change which the presence of the SNP marker makes to the mobility of the polynucleotide or protein during gel electrophoresis. In the case of a polynucleotide single-stranded conformation SNP marker (SSCP) or denaturing gradient gel electrophoresis (DDGE) analysis may be used.

In another method of detecting the SNP marker a polynucleotide comprising the polymorphic region is sequenced across the region which contains the SNP marker to determine the presence of the SNP marker.

Polynucleotides

The invention also provides a polynucleotide which comprises a breed-specific SNP. Preferably the SNP position is any one of those identified in any one of SEQ ID NO:s 1 or 4 to 23. The polynucleotide is typically at least 10, 15, 20, 30, 50, 100, 200 or 500 bases long, such as at least or up to 1 kb, 10 kb, 100 kb, 1000 kb or more in length. The polynucleotide will typically comprise flanking nucleotides on one or both sides of (5′ or 3′ to) the SNP position, for example at least 2, 5, 10, 15 or more flanking nucleotides in total or on each side. Thus such flanking sequences of the 5′ or 3′ side may be fully or partially identical to or fully or partially complementary to (i.e. have homology with) either all or part of the flanking 5′ and/or 3′ sequences identified in any one of SEQ ID NO:s 1 or 4 to 23.

The polynucleotide may differ to the sequences identified in any one of SEQ ID NO:s 1 or 4 to 23 by less than 30, 20, 10, 5, 3 or 2 substitutions and/or insertions and/or deletions in sequence, apart from at the polymorphic position. Typically, the polynucleotide will be at least 95%, preferably at least 99%, even more preferably at least 99.9% identical to the sequence comprising the SNP position as identified in any one of SEQ ID NO:s 1 or 4 to 23. Such numbers of substitutions and/or insertions and/or deletions and/or percentage homology may be taken over the entire length of the polynucleotide or over 50, 30, 15, 10 or less flanking nucleotides in total or on each side.

The polynucleotide may be RNA or DNA, including genomic DNA, synthetic DNA or cDNA. The polynucleotide may be single or double stranded. The polynucleotide may comprise synthetic or modified nucleotides, such as methylphosphonate and phosphorothioate backbones or the addition of acridine or polylysine chains at the 3′ and/or 5′ ends of the molecule.

A polynucleotide of the invention may be used as a primer, for example for PCR, or a probe. A polynucleotide or polypeptide of the invention may carry a revealing label. Suitable labels include radioisotopes such as ³²P or 35S, fluorescent labels, enzyme labels or other protein labels such as biotin.

The invention also provides expression vectors that comprise polynucleotides of the invention and are capable of expressing a polypeptide of the invention. Such vectors may also comprise appropriate initiators, promoters, enhancers and other elements, such as for example polyadenylation signals which may be necessary, and which are positioned in the correct orientation, in order to allow for protein expression. Thus the coding sequence in the vector is operably linked to such elements so that they provide for expression of the coding sequence (typically in a cell). The term “operably linked” refers to a juxtaposition wherein the components described are in a relationship permitting them to fimction in their intended manner.

The vector may be for example plasmid, virus or phage vector. Typically the vector has an origin of replication. The vector may comprise one or more selectable marker genes, for example an ampicillin resistance gene in the case of a bacterial plasmid or a resistance gene for a fingal vector. Vectors may be used in vitro, for example for the production of DNA or RNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR).

The vector may further include sequences flanking the polynucleotide giving rise to polynucleotides which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

The polynucleotide may be a probe or primer which is capable of selectively binding to a breed-specific SNP. Preferably the probe or primer is capable of selectively binding to a SNP position as identified in any one of SEQ ID NO:s 1 or 4 to 23. The probe or primer more preferably comprises a fragment of a nucleic acid sequence of any one of SEQ ID NO:s 1 or 4 to 23 which comprises the SNP position. The invention thus provides a probe or primer for use in a method according to the invention, which probe or primer is capable of selectively detecting the presence of a breed-specific SNP. Preferably the probe is isolated or recombinant nucleic acid. Preferably it is at least 10, 15, 20 or 25 bases in length. It may correspond to or be antisense to the sequences set out in any one of SEQ ID NO:s 1 or 4 to 23. The probe may be immobilized on an array, such as a polynucleotide array.

The polypeptides, polynucleotides, vectors, cells or antibodies of the invention may be present in an isolated or substantially purified form. They may be mixed with carriers or diluents which will not interfere with their intended use and still be regarded as substantially isolated. They may also be in a substantially purified form, in which case they will generally comprise at least 90%, e.g. at least 95%, 98% or 99%, of the proteins, polynucleotides, cells or dry mass of the preparation.

It is understood that any of the above features that relate to polynucleotides and proteins may also be a feature of the other polypeptides and proteins mentioned herein, such as the polypeptides and proteins used in the screening and therapeutic aspects of the invention. In particular such features may be any of the lengths, modifications and vectors forms mentioned above.

Homologues

Homologues of polynucleotide or protein sequences are referred to herein. Such homologues typically have at least 70% homology, preferably at least 80, 90%, 95%, 97% or 99% homology, for example over a region of at least 15, 20, 30, 100 more contiguous nucleotides or amino acids. The homology may be calculated on the basis of nucleotide or amino acid identity (sometimes referred to as “hard homology”).

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information. This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al, supra). These initial neighborhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differs by at least 1, 2, 5, 10, 20 or more mutations (which may be substitutions, deletions or insertions of nucleotide or amino acids). These mutations may be measured across any of the regions mentioned above in relation to calculating homology. In the case of proteins the substitutions are preferably conservative substitutions. These are defined according to the following Table. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Antibodies

The invention also provides antibodies specific for a polypeptide of the invention. The antibodies include those which are specific for proteins which have a breed-specific SNP, such as any of the SNPs mentioned herein, but which do not bind to protein sequences that do not contain the breed-specific SNP. The antibodies of the invention are for example useful in purification, isolation or screening methods involving immunoprecipitation techniques.

Antibodies may be raised against specific epitopes of the polypeptides of the invention. An antibody, or other compound, “specifically binds” to a polypeptide when it binds with preferential or high affinity to the protein for which it is specific but does substantially bind not bind or binds with only low affinity to other polypeptides. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanized antibodies.

Antibodies may be used in a method for detecting polypeptides of the invention in a biological sample (such as any such sample mentioned herein), which method comprises:
I providing an antibody of the invention;
II incubating a biological sample with said antibody under conditions which allow for the formation of an antibody-antigen complex; and
III determining whether antibody-antigen complex comprising said antibody is formed.

Antibodies of the invention can be produced by any suitable method. Means for preparing and characterizing antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”. The fragment may be any of the fragments mentioned herein (typically at least 10 or at least 15 amino acids long) and comprise a SNP marker (such as any of the SNP markers mentioned herein).

A method for producing a polyclonal antibody comprises immunizing a suitable host animal, for example an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalizing cells which produce the desired antibody. Hybridoma cells may be produced by fusing spleen cells from an inoculated experimental animal with tumor cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunization of human lymphocytes, followed by transformation of the lymphocytes with Epstein-Barr virus.

For the production of both monoclonal and polyclonal antibodies, the experimental animal is suitably a goat, rabbit, rat, mouse, guinea pig, chicken, sheep or horse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

Detection kit

The invention also provides a kit that comprises means for determining the nucleotide present at one or more breed specific genomic SNP positions in a dog. In particular, such means may include a specific binding agent, probe, primer, pair or combination of primers, or antibody, including an antibody fragment, as defined herein which is capable of detecting or aiding detection of a breed-specific SNP. The primer or pair or combination of primers may be sequence specific primers which only cause PCR amplification of a polynucleotide sequence comprising a particular nucleotide at the SNP position, as discussed herein. The means for determining nucleotide present at one or more breed specific SNP positions (such as the binding agent, probe, primer or antibody as discussed herein) may be provided in containers that are labeled with the breed for which the SNP is specific. The kit may further comprise buffers or aqueous solutions.

The kit may additionally comprise one or more other reagents or instruments which enable any of the embodiments of the method mentioned above to be carried out. Such reagents or instruments may include one or more of the following: a means to detect the binding of the agent to the SNP, a detectable label such as a fluorescent label, an enzyme able to act on a polynucleotide, typically a polymerase, restriction enzyme, ligase, RNAse H or an enzyme which can attach a label to a polynucleotide, suitable buffer(s) or aqueous solutions for enzyme reagents, PCR primers which bind to regions flanking the SNP position as discussed herein, a positive and/or negative control, a gel electrophoresis apparatus, a means to isolate DNA from a sample, a means to obtain a sample from the individual, such as swab or an instrument comprising a needle, or a support comprising wells on which detection reactions can be carried out. The kit may be, or include, an array such as a polynucleotide array comprising the specific binding agent, preferably a probe, of the invention. The kit typically includes a set of instructions for using the kit.

Customized Dog Food

In one aspect, the invention relates to a customized diet for a dog based on its nutritional needs, as determined by its breed inheritance. Such a food may be in the form of, for example, wet pet foods, semi-moist pet foods, dry pet foods and pet treats. Wet pet food generally has a moisture content above 65%. Semi-moist pet food typically has a moisture content between 20-65% and can include humectants and other ingredients to prevent microbial growth. Dry pet food, also called kibble, generally has a moisture content below 20% and its processing typically includes extruding, drying and/or baking in heat. Pet treats can be semi-moist, chewable treats; dry treats; chewable bones; baked, extruded or stamped treats; or other types of treats which are known in the art.

The ingredients of a dry pet food generally include cereal, grains, meats, poultry, fats, vitamins and minerals. The ingredients are typically mixed and put through an extruder/cooker. The product is then typically shaped and dried, and after drying, flavors and fats may be coated or sprayed onto the dry product.

All pet food is required to provide a certain level of nutrients. For example, the Association of American Feed Control Officials (AAFCO) and the Pet Food Institute have established nutrient profiles for dog foods, based on commonly used ingredients. These established profiles are called the “AAFCO dog food nutrient profiles”. Under these regulations, dog foods must be formulated to contain concentrations of nutrients that meet all minimum levels and not to exceed the maximum levels as determined by AAFCO.

The AAFCO nutritional guideline provides adequate nutrition but may not provide the dog with optimal nutrition. For this reason, dog food formulations have been developed which meet the specific needs of various dog breeds or breed categories. For example, a breed specific diet for the Bedlington Terrier typically comprises a dry product containing 18% protein, 18% fat, 7% ash, 2% fiber, and a wet product containing 8% protein, 5% fat, 1% ash and 2% fiber. The ingredients used are typically chicken, cereals and byproducts, and supplementary vitamins, minerals, and amino acids.

Accordingly, the present invention enables the preparation of customized dog food, wherein one or more nutritional requirements of the dog is determined by a method of the invention, a customized dog food formulation that corresponds to the nutritional requirements of the dog is generated, and a dog food according to the customized dog food formulation is prepared. The preparation of customized dog food may be carried out by electronic means, for example by using a computer system.

The dog food formulation may be customized according to the caloric, protein, fat, carbohydrate, fiber, vitamin or mineral requirements of the dog, as discussed herein. For example, the dog food formulation may be customized to provide the correct amounts or ratio of essential fatty acids such as omega-6 and omega-3 fatty acids. The main sources of omega-6 fatty acids are plants such as sunflower, soyabean oil, safflower and evening primrose oil, whereas omega-3 fatty acids are mainly found in linseed and marine sources, for example canola oil and salmon oil.

In one embodiment, the customized dog food formulation comprises components suitable for the breed(s) which have contributed to the genetic breed inheritance of the dog, and does not comprise components that are not suitable for the breed(s) which have contributed to the genetic breed inheritance of the dog.

Accordingly, in one aspect of the invention, the customized food does not contain ingredients which are poorly tolerated or cause allergies, are abnormally processed or stored, or contribute to diseases or conditions typically suffered by the breed(s) which have contributed to the genetic breed inheritance of the dog. In another aspect of the invention, the customized food contains ingredients which are commonly lacking in, or have nutritional or medical benefits for the breed(s) which have contributed to the genetic breed inheritance of the dog.

For example, the customized food may be formulated so that it does not contain ingredients that are poorly tolerated or cause allergies, for example gluten-containing grains such as wheat, particular protein sources such as animal proteins, milk (lactose), eggs, soy, peanuts, shellfish, fruits or tree nuts. The customized food formulation may further exclude ingredients that are abnormally processed or stored or contribute to diseases or conditions, for example copper, saturated fats and salt.

In another embodiment, the customized food may be formulated to include functional ingredients that help prevent disease or have other beneficial effects for the dog, such as: vitamins, minerals, cocoa flavanols, other plant flavanols, lycopene, curcumin, minerals, trace metals, Echineacea, phosphatidyl serine, L-arginine, ginseng, psyllium, prebiotics, probiotics, phyto-oestrogens, phyto-chemicals, soluble fiber, PUFAs, phospholipids, omega-6 and omega-3 fatty acids.

The present invention also relates to a method of providing a customised dog food, comprising providing to: (a) the dog's owner, the person responsible for feeding the dog or a vet; or (b) the dog; a food which contains components suitable for the breed(s) which have contributed to the genetic breed inheritance of the dog, and which does not contain components that are not suitable for the breed(s) which have contributed to the genetic breed inheritance of the dog, wherein the breed inheritance of the dog has been identified by determining the nucleotide present at one or more SNP positions in the dog's genome.

In another aspect of the invention, there is provided a method of feeding a dog comprising feeding a mixture of foods that have been formulated for specific breeds or breed categories, based on the dog's genetic breed inheritance. For example, an outbred dog that has breed-specific markers for two different breeds could be fed a mixture of breed-specific food formulations for those two breeds. A dog that showed the presence of breed-specific markers from one or more breeds in a particular category could be fed food that had been formulated for that breed category. It may be that the nutritional requirements of one of the breeds from which a crossbred or outbred dog is derived is dominant over the one or more other breeds represented in the dog. In that case, the customised food may be tailored to meet the requirements of the dominant breed. Alternatively, the food may be customised according to the proportion of genetic inheritance from each breed represented.

Disease

The invention provides a method of treating a dog for a disease that occurs in a dog breed, comprising identifying a disease susceptibility by a method of the invention, and administering to the dog an effective amount of a therapeutic agent which prevents or treats the disease. The therapeutic agent is typically a drug such as an anti-inflammatory, antibiotic, vasodilator, calcium blocker, vaccine, insecticide or hormone. In the case of behavioral problems, the therapeutic agent may be a drug such as an antihistamine, tranquilizer, mood stabilizer, anticonvulsant, progestin, antidepressant, anxiolytic or narcotic.

The therapeutic agent may be administered in various manners such as orally, intracranially, intravenously, intramuscularly, intraperitoneally, intranasally, intrademally, and subcutaneously. The pharmaceutical compositions that contain the therapeutic agent will normally be formulated with an appropriate pharmaceutically acceptable carrier or diluent depending upon the particular mode of administration being used. For instance, parenteral formulations are usually injectable fluids that use pharmaceutically and physiologically acceptable fluids such as physiological saline, balanced salt solutions, or the like as a vehicle. Oral formulations, on the other hand, may be solids, e.g. tablets or capsules, or liquid solutions or suspensions.

The amount of therapeutic agent that is given to a dog will depend upon a variety of factors including the condition being treated, the nature of the dog under treatment and the severity of the condition under treatment. A typical daily dose is from about 0.1 to 50 mg per kg, preferably from about 0.1 mg/kg to 10 mg/kg of body weight, according to the activity of the specific inhibitor, the age, weight and conditions of the subject to be treated, the type and severity of the disease and the frequency and route of administration. Preferably, daily dosage levels are from 5 mg to 2 g.

Bioinformatics

The sequences of the breed-specific SNPs may be stored in an electronic format, for example in a computer database. Accordingly, the invention provides a database comprising information relating to breed-specific genomic SNPs. The database may include further information about the SNP, for example the level of association of the SNP marker with the breed or the frequency of the SNP in the breed. The database may optionally comprise information relating to the nutritional requirements, disease susceptibility or behavioral characteristics of the breeds for which the SNPs are specific. In one aspect of the invention, the database further comprises information regarding the food components which are suitable and the food components which are not suitable for the breeds for which the SNPs are specific.

A database as described herein may be used to determine the breed inheritance of a dog. Such a determination may be carried out by electronic means, for example by using a computer system (such as a PC). Typically, the determination will be carried out by inputting genetic data from the dog to a computer system; comparing the genetic data to a database comprising information relating to breed-specific genomic SNPs; and on the basis of this comparison, determining the nucleotide present at one or more breed-specific SNP positions, thereby identifying the breed inheritance of the dog. A method for determining the nutritional requirements, disease susceptibility or behavioral characteristics of a dog according to the invention may also be carried out by electronic means, for example by using a computer system (such as a PC). Typically, the method will comprise inputting data of the breed-specific genomic SNPs present in the dog to a computer system; comparing this data to a database which comprises information relating to breed-specific genomic SNPs and the nutritional requirements, disease susceptibility or behavioral characteristics of the breeds; and determining on the basis of the comparison the nutritional requirements, disease susceptibility or behavioral characteristics of the dog.

The invention also provides a computer program comprising program code means for performing all the steps of a method of the invention when said program is run on a computer. Also provided is a computer program product comprising program code means stored on a computer readable medium for performing a method of the invention when said program is run on a computer. A computer program product comprising program code means on a carrier wave that, when executed on a computer system, instruct the computer system to perform a method of the invention is additionally provided.

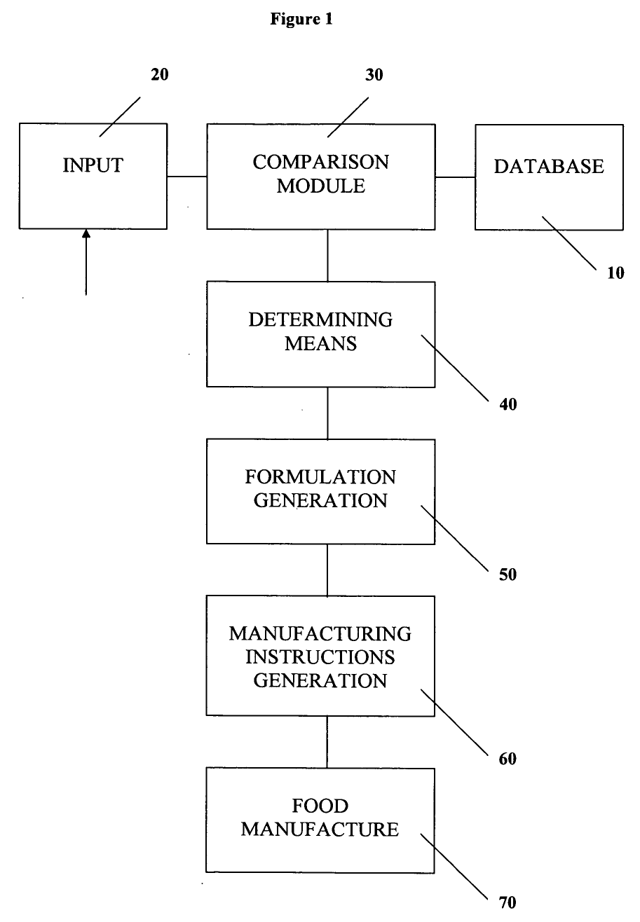

As illustrated in FIG. 1, the invention also provides an apparatus arranged to perform a method according to the invention. The apparatus typically comprises a computer system, such as a PC. In one embodiment, the computer system comprises: means 20 for receiving data of breed-specific genomic SNP markers; a module 30 for comparing the data with a database 10 comprising information relating to breed-specific genomic SNPs and optionally the nutritional requirements, disease susceptibility or behavioral characteristics of the breeds; and means 40 for determining on the basis of said comparison the breed inheritance and optionally the nutritional requirements, disease susceptibility or behavioral characteristics of the dog.

Food Manufacturing

In one embodiment of the invention, the manufacture of a customized dog food may be controlled electronically. Typically, the nutritional requirements of the dog may be processed electronically to generate a customized dog food formulation. The customized dog food formulation may then be used to generate electronic manufacturing instructions to control the operation of food manufacturing apparatus. The apparatus used to carry out these steps will typically comprise a computer system, such as a PC, which comprises means 50 for processing the nutritional requirement information to generate a customized dog food formulation; means 60 for generating electronic manufacturing instructions to control the operation of food manufacturing apparatus; and a food product manufacturing apparatus 70.

The food product manufacturing apparatus used in the present invention typically comprises one or more of the following components: container for dry pet food ingredients; container for liquids; mixer; former and/or extruder; cut-off device; cooking means (e.g. oven); cooler; packaging means; and labeling means. A dry ingredient container typically has an opening at the bottom. This opening may be covered by a volume-regulating element, such as a rotary lock. The volume-regulating element may be opened and closed according to the electronic manufacturing instructions to regulate the addition of dry ingredients to the pet food. Dry ingredients typically used in the manufacture of pet food include corn, wheat, meat and/or poultry meal. Liquid ingredients typically used in the manufacture of pet food include fat, tallow and water. A liquid container may contain a pump that can be controlled, for example by the electronic manufacturing instructions, to add a measured amount of liquid to the pet food.

In one embodiment, the dry ingredient container(s) and the liquid container(s) are coupled to a mixer and deliver the specified amounts of dry ingredients and liquids to the mixer. The mixer may be controlled by the electronic manufacturing instructions. For example, the duration or speed of mixing may be controlled. The mixed ingredients are typically then delivered to a former or extruder. The former/extruder may be any former or extruder known in the art that can be used to shape the mixed ingredients into the required shape. Typically, the mixed ingredients are forced through a restricted opening under pressure to form a continuous strand. As the strand is extruded, it may be cut into pieces (kibbles) by a cut-off device, such as a knife. The kibbles are typically cooked, for example in an oven. The cooking time and temperature may be controlled by the electronic manufacturing instructions. The cooking time may be altered in order to produce the desired moisture content for the food. The cooked kibbles may then be transferred to a cooler, for example a chamber containing one or more fans.

The pet food manufacturing apparatus may comprise a packaging apparatus. The packaging apparatus typically packages the pet food into a container such as a plastic or paper bag or box. The apparatus may also comprise means for labeling the pet food, typically after the food has been packaged. The label may provide information such as: ingredient list; nutritional information; date of manufacture; best before date; weight; and breed(s) or breed category or sub-group for which the food is suitable. In one embodiment of the invention, there is provided a labeled dog food product, wherein the food product is customized for one or more breed(s) and the label provides an indication of one or more breed specific genomic SNP marker(s) present in said breed(s).

Breeding Method

The present invention provides a method of determining the genetic breed background of a dog, which comprises determining the nucleotide present at one or more SNP positions in the dog and identifying therefrom the genetic breed inheritance of the dog. In one aspect of the invention, the terms “genetic breed background” and “genetic breed inheritance” relate to a dog's breed. Accordingly, in one embodiment the invention provides a method of determining the breed of a dog. In this case, the breed-specific SNPs are used to distinguish between dogs of different breeds.

In another aspect of the invention, the terms “genetic breed background” and “genetic breed inheritance” relate to the dog's genetic ancestry within a particular breed. The breed-specific SNPs present in an individual dog will be derived from either the maternal or paternal line used to breed that dog. Accordingly, it is possible to use a “breed-specific SNP” as defined herein to distinguish between dogs within a single breed in order to determine how closely related they are. Therefore, the present invention provides a method of determining the degree of relatedness between two dogs of the same breed, which comprises comparing the genetic breed inheritance of a dog with another dog of the same breed in order to determine the degree of relatedness between the two or more dogs. Preferably the dogs are purebred dogs. Typically the genetic breed inheritance of each dog is determined by identifying the nucleotide present at one or more SNP positions in said dog, as described herein.

The degree of relatedness may be determined from the number of nucleotides at breed-specific SNP positions that the dogs have in common. For example, two dogs of the same breed may have from 0 to 100% of the breed-specific SNPs tested in common, for example from 10 to 90%, from 20 to 80%, from 30 to 70% or from 40 to 60%. Therefore two dogs may have at least 10%, 20%, 30%, 40%, 50%, 60%, 70%, 80% or 90% of the breed-specific SNPs tested in common. The percentage of tested breed-specific SNPs in common between two dogs may be used as a measure of their degree of relatedness.

Most purebred dogs of breeds recognized by all-breed club registries are controlled by “closed studbooks”. A studbook is typically the official registry of approved dogs of a given breed kept by, for example, a breed association or kennel club. It is generally termed a “closed” studbook if dogs can only be added if their parents were both registered. Most breeds have closed studbooks, resulting in inbreeding, as genetic diversity cannot be introduced from outside the existing population. In a number of breeds recognized by kennel clubs this has resulted in high incidences of genetic diseases or disorders and other problems such as reduced litter sizes, reduced lifespan and inability to conceive naturally.

In order to avoid the problems associated with inbreeding, it would be advantageous to select dogs for breeding within a particular breed that are more distantly related to each other compared to dogs that are more closely related. This problem is solved by the use of breed-specific SNPs that can be used to determine the degree of relatedness between two or more dogs of the same breed, in order to inform breeding of purebred dogs.

Accordingly, the invention provides a method of selecting one or more dogs for breeding with a subject dog, the method comprising:

(a) comparing the genetic breed inheritance of the subject dog with the genetic breed inheritance of each dog in a test group of two or more dogs of the same breed and of the opposite sex to the subject dog;

(b) determining from the comparison the degree of relatedness between the subject dog and each dog in the test group; and

(c) selecting one or more dogs from the test group for breeding with the subject dog.

The test group may consist of at least 2, 3, 4, 5, 10, 15, 20, 25, 30, 50, 75, 100 or 200 different dogs of the same breed, for example from 2 to 100, from 5 to 70 or from 10 to 50 dogs. The dogs are typically selected from the test group on the basis of their relatedness to the subject dog (i.e. the dog to be bred from). Preferably the dog or dogs selected from the test group are the most distantly related (i.e. have the lowest degree of relatedness) within the test group of dogs. This is in order to increase or maintain genetic diversity within the breed, and to reduce the likelihood of problems relating to inbreeding arising within the offspring.

In one embodiment of the invention, the one dog within the test group that is most distantly related (i.e. has the lowest degree of relatedness) to the subject dog is selected for breeding with the subject dog. In another embodiment, a number of the most distantly related dogs within the test group are selected for breeding with the subject dog. For example, at least 2, 3, 4, 5, 10, 15 or 20 dogs in the test group may be selected. A further selection may then be made from the group of selected dogs based on other factors, for example geographical location, age, breeding status, medical history, disease susceptibility or physical characteristics. The genetic breed background of the subject dog and the dogs in the test group may be already known or may be determined by a method of the present invention.

The invention thus provides a method of recommending one or more suitable dogs for breeding with a subject dog. The recommendation may be made to the subject dog's owner or carer, a veterinarian, dog breeder, kennel club or breed registry. The invention also relates to a method of breeding dogs, wherein the genetic breed background of a subject dog is compared to a dog of the opposite sex within the same breed in order to determine the degree of relatedness between the two dogs before breeding them together. The genetic breed background of a dog may be stored in an electronic format, for example in a computer database. Accordingly, the invention provides a database comprising information relating to the genetic breed background and sex of one or more dogs of the same breed. The database may include further information about the dog, for example the dog's breeding status, age, geographical location, medical history, disease susceptibility or physical characteristics. The database will typically further comprise a unique identifier for each dog, for example the dog's registered name. The database may be accessed remotely, for example using the internet.

A method of selecting one or more dogs for breeding with a subject dog according to the invention may also be carried out by electronic means, for example by using a computer system (such as a PC). Typically, the method will comprise inputting data of the genetic breed inheritance of the subject dog to a computer system; comparing this data to a database which comprises information relating to the genetic breed inheritance and sex of each dog in a test group of two or more dogs of the same breed; on the basis of the comparison, determining the degree of relatedness between the subject dog and each dog in the test group; and selecting one or more dogs from the test group for breeding with the subject dog. Selection of dogs that are suitable for breeding with the subject dog is primarily based on the degree of relatedness between the test dog and the subject dog. However, the selection may also take into account other factors such as geographical location, age, breeding status, medical history, disease susceptibility or a physical characteristic of the dogs in the test group.

The invention also provides a computer program comprising program code means for performing all the steps of a method of the invention when said program is run on a computer. Also provided is a computer program product comprising program code means stored on a computer readable medium for performing a method of the invention when said program is run on a computer. A computer program product comprising program code means on a carrier wave that, when executed on a computer system, instruct the computer system to perform a method of the invention is additionally provided.

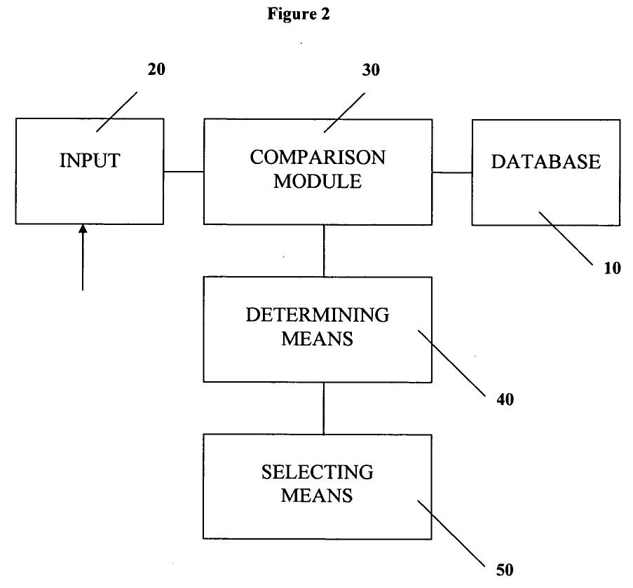

As illustrated in FIG. 2, the invention also provides an apparatus arranged to perform a method according to the invention. The apparatus typically comprises a computer system, such as a PC. In one embodiment, the computer system comprises: means 20 for receiving data of genetic breed inheritance from a subject dog; a module 30 for comparing the data with a database 10 comprising information relating to the genetic breed inheritance of one or more dogs in a test group and optionally their sex, age and geographical location; means 40 for determining on the basis of said comparison the degree of relatedness between the subject dog and at least one test dog; and means (50) for selecting one or more test dogs for breeding with the subject dog.

The invention is illustrated by the following Examples:

### EXAMPLE 1

DNA Samples

Buccal cells were collected from 72 dogs of 16 different breeds by scraping the inside cheek six times with a sterile cytology brush (Rocket Medical, Cat No. R57483), ensuring that the animal providing the sample had not consumed any food or drink for 30min prior to sample collection. The brushes were then replaced in their individual wrappers and left to dry for a minimum of 2 hours at room temperature. DNA was extracted using standard techniques (Qiagen's QIAamp DNA Blood Mini Kit, Cat No. 51104) following the Buccal Swab Spin protocol. The DNA was then stored at −20° C.

PCR Amplifications

The primers used were designed to amplify products ranging from 200-600 bp in length. The primers were designed by eye, and were made to be approximately 20 bp in length, with approximately 50% G/C, 50% A/T ratio. These were ordered from Sigma-Genosys, desalted, and at 0.025 μM synthesis scale. 12.5 ng of genomic dog DNA (Gibco, Cat. 69234) was added to 12.5 ng of DNA from each dog and was amplified up in 25 μl PCR reactions with Eurogentec HotGoldstar PCR mastermix (PK-0073-02). Reactions contained 1.5 mM MgCl₂ and 25 pmol of each primer. Thermal cycling was performed using a Hybaid MBS 0.2S PCR machine using the following cycling conditions: an initial incubation of 95° C. for 10 min, followed by 30 cycles of 95° C. for 30 sec, 60° C. for 45 sec and 72° C. for 90 sec. This was followed by a final extension step of 72° C. for 5 min.

Single Nucleotide Polymorphism Identification

The base sequence of the wild-type amplicon was manually inputted into the Transgenomic Wave Machine, and the PCR products run according to the manufacturers directions as described in the WAVEMAKER Software Manual, Transgenomic Inc. 1999, version 2.0 October 1999. Chromatograms were examined for the presence of additional peaks indicating the presence of a single nucleotide polymorphism in the sample. The PCR amplification described above was repeated on the DNA samples indicated to have SNPs present, with the following change: 25 ng of the test DNA sample was added to the PCR reaction, and no other DNA was added. The PCR products were then purified using a Qiagen PCR Purification Kit (Cat No. 28104), following the method Qiaquick PCR Purification using a microcentrifuge.

DNA Sequencing

Cycle sequencing was performed using 25 fmol of purified PCR product with the CEQ 2000 Dye Terminator Cycle Sequencing with Quick Start Kit (Beckman Coulter, P/N 608120). 20 μt reactions were prepared as described in the manufacturers directions using the same primers used in the PCR step, and were subjected to 30 cycles at 95° C. for 20 sec, 60° C. for 20 sec and 72° C. for 4 min. Following these cycles, the samples were subjected to ethanol precipitation, and were evaporated to dryness using a vacuum pump for approximately 40 min. The samples were then resuspended in 40 μl of deionized formamide and a drop of mineral oil was placed on top. The samples were then run on a Beckman CEQ 2000 Sequencer using the LFR capillary method. SNPs were called using the CEQ2000XL DNA Analysis System Software Version 4.3.9, and were confirmed using the reverse traces.

Results

SNP was identified in the mast cell chymase gene of the breed English Mastiff at position 5375. The SNP is shown as underlined in the sequence below (SEQ ID NO: 1). The position 5375 is defined using standard nomenclature as can be seen in accession NCBI Ref U89607. The following primers were used for PCR amplification:

| | |
|---|---|
| Forward Primer (5260 bp-5279 bp) | ACT CCA CTT CAC CTC CAG C |
| Reverse Primer (5600 bp-5620 bp) | AGA GAT CCT GCC ACC TTG C |

| | | |
|---|---|---|
| ACTCCACTTCACCTCCAGCAAAACAGAGCATAACTTGGAAGAAACATCTG | 50 | (SEQ ID NO: 1) |
| ACTCCACTTCACCTCCAGCAAAACAGAGCATAACTTGGAAGAAACATCTG | | |
| ATCAGAAAGATAGCCTAATATGGGAGAAGAAAAACATGACCACATAGTTC | 100 | |
| ATCAGAAAGATAGCCTAATATGGGAGAAGAAAAACATGACCACATAGTTC | | |
| CTGTGGTTACCAGCCCAGCCCTTGGCTCATTGCTGGAGTTATAAAACCCA | 150 | |
| CTGTGGTTACCAGCCTAGCCCTTGGCTCATTGCTGGAGTTATAAAACCCA | | |
| AGACCAGAAAATAGAAGCAGCATCTGCCCAGGGCAGCCTCACTGAGAAGA | 200 | |
| AGACCAGAAAATAGAAGCAGCATCTGCCCAGGGCAGCCTCACTGAGAAGA | | |
| TGCATTGTCTTCCTCTCACCCTGCTGCTCCTTCTCCTATGTTCCAGAGCA | 250 | |
| TGCATTGTCTTCCTCTCACCCTGCTGCTCCTTCTCCTATGTTCCAGAGCA | | |
| GAAGCTGGTGAGTCTTGGGATCCTTCCCCCTGGAAACGGCAGGATCAGCA | 300 | |
| GAAGCTGGTGAGTCTTGGGATCCTTCCCCCTGGAAACGGCAGGATCAGCA | | |
| CCCCAAAACCAAGTTTAGTCTGAATATAGCTGACTCATAAGCAAGGTGGC | 350 | |
| CCCCAAAACCAAGTTTAGTCTGAATATAGCTGACTCATAAGCAAGGTGGC | | |
| AGGATCTCTCT | | |
| AGGATCTCTCT | | |

TABLE 2

| | | | SNP and |
|---|---|---|---|
| SNP | Breed | NCBI Ref for gene | flanking sequences |
| Mast cell | English | U89607 | SEQ ID NO: 1 |
| chymase/C5375T | Mastiff | | |

This SNP was found in English Mastiff dogs, and not in any of the other 15 different breeds tested. Hence the mast cell chymase SNP identified above is unique to this breed.

### EXAMPLE 2

DNA Samples

Dog genomic DNA was acquired from various sources. In total, 51 dogs were included in the study: 5 German Shepherds, 6 Rottweilers, 6 Daschunds, 6 Cocker Spaniels, 6 Golden Retrievers, 2 Poodles, 2 Beagles, 6 Yorkshire Terriers, 6 Shih Tzus and 6 Labradors.

Obtaining Sequence

In order to obtain the canine sequence for a gene of interest it was necessary to firstly acquire the cDNA sequence of the human form. A search was carried out for the gene at the ensembl website for searching under the gene database. To obtain the DNA sequence for the same gene in the dog, the ncbi webpage was accessed. A search was carried out, searching the nucleotide database for canis familiaris. The original human cDNA sequence was then accessed from the transcript information gained through ensembl and this sequence was copied into the cross-species megablast. Canis familiaris WGS (whole genome sequence) was chosen in the database field. A blast search was then carried out. From the blast results all those alignments with a score over 200 were selected.

The gene sequence was then edited before primer design could take place. A blast search was carried out on the sequence using blastn (nucleotide-nucleotide blast). The results of this blast search highlighted the repeat regions within the sequence. An additional blast search using blastx (translated query vs protein database) was used to determine the position of exons in the sequence. Blast results were checked to ensure that firstly they were actually relevant to the gene of interest and also that they were in a positive reading frame. Only those results with a high % identity were marked on the sequence as exons.

Primer Design

Primers were manually designed along the contig at 600 bp spacing. The forward primer of each amplicon was located approximately 50 bp before the reverse primer of the previous amplicon. Primer design was concentrated around exonic regions and away from repeat regions. Primers were approximately 20 bases long with a melting temperature between 56° C. and 64° C. The primers were ordered desalted at a synthesis scale of 0.025 μM (Sigma-Genosys).

PCR Amplification

PCR reactions were carried using 25 pmol of each primer, 25 ng of commercial dog genomic DNA (Novegen, Cat. No 69234) and 12.5 μl of Eurogentec HotGoldstar PCR mastermix containing a red loading dye and 1.5 mM MgCl (PK-0073-02R). Thermal cycling was performed using a Hybrid MBS 0.2S PCR machine using the following cycling conditions: incubation at 95° C. for 10 mins, 10 cycles of 95° C. for 30 seconds, followed by 64° C. (minus 1° C. per cycle) for 45 seconds and 72° C for 90 seconds, and 28 cycles of 95° for 30 seconds, 55° C. for 45 sec and 72° C. for 90 seconds. 5ul of each PCR sample and 1 ul Gelstar nucleic acid gel stain (BioWhittaker Molecular Applications, Cat. No 50535) was run on a 2% agarose gel (Invitrogen, Cat. No 15510-027) at 100 mV to check for the presence of product. Primers used in successful PCR reactions were plated into forward and complementary reverse plates at a concentration of 25 pmol. Samples were quantified with the Nanodrop Spectrophotometer using 1 μl of purified DNA. Analysis was carried out using Nanodrop 2.4.7a DNA-50 software. DNA was diluted to 100 ng/μl.

Sequencing

All sets of DNA were amplified using each primer pair, under the same conditions as stated above. The PCR reactions were purified and a sample of purified product was sequenced. Both forward and reverse sequence traces were generated using the original primers for the reaction.

PolyPhred Analysis

In order to identify SNPs (single nucleotide polymorphisms) the PolyPhred computer programme was employed. PolyPhred automatically detects the presence of heterozygous single nucleotide substitutions by comparing the pattern of the fluorescence dye incorporation between traces (Nickerson et al. Nucleic Acids Research 25 (14):2745-2751, 1997). PolyPhred is not used alone but in conjuction with Phred automated base calling, Phrap sequence assembly and Consed sequence assembly editing. The output from PolyPhred was then reformatted for the genetic algorithm software (G-max).

Gmax

The objective of the next stage was to derive a way of determining breed status using a pattern of SNPs found via sequencing. The Gmax software, accessible by the website, uses a genetic algorithm to extract such patterns from large data sets. The pattern is extracted in the form of a rule. Each rule is expressed as a Boolean formula, where “&” is “AND”, “|” is “OR”, and “!” is “NOT”.

Gmax was used to screen thousands of SNPs to find a combination of a smaller number that define the breed well. For example, using 5 SNPs from a possible 1000, there will be 10¹⁷ possible combinations to search through. Randomly picking rules to fit the data would not work very well. However, a fitness test can determine how well a random rule performs at separating the data and comparing it to how close to a solution it is. If small changes are made to the rule and retest a second score for a new rule is generated. A continuation of this process will evolve the rule. This way of working is called ‘hill climbing’. The problem with hill climbing is that for complex fitness tests there are local maximums. If a local maximum is reached then the overall solution will not be found. A genetic algorithm solves this problem by keeping a large population of rules and applying a form of Darwinian evolution.

Results

Rules were generated for 4 breeds, namely Cocker Spaniel, Shih Tzu, Doberman and Golden Retriever. Tables 3 to 6 show the rules for each breed in the form of a Boolean formula. Table 7 shows the sequences surrounding each SNP, and which bases may be present at each polymorphic position (options). The full name of each gene is abbreviated as follows:
FCGR2A: FC gamma receptor RIIa;
FCGR3B: FC gamma receptor RIIb;
RAGE: receptor for advanced glycation end product; and

PAI1: plasminogen activator inhibitor 1.

TABLE 3
Cocker Spaniel

| | A | | B | | C | |
|---|---|---|---|---|---|---|
| Boolean formula | SNP | Allele | SNP | Allele | SNP | Allele |
| A & ! B | RAGE_8Kb_6000 | AA | RAGE_8Kb_6002 | AA | | |
| ! (A \| (B & C)) | RAGE_8Kb_6002 | AA | RAGE_8Kb_5959 | TT | FCGR3B_7.42Kb_5238 | CC |
| ! (A \| B) | RAGE_8Kb_6002 | AA | PAI1_10KB_2979 | TT | | |
| ! (A \| B) | RAGE_8Kb_6002 | AA | PAI1_10KB_3317 | GG | | |
| ! (A \| B) | RAGE_8Kb_6002 | AA | RAGE_5KB_4330 | AA | | |

TABLE 4
Shih Tzu

| | A | | B | | C | |
|---|---|---|---|---|---|---|
| Boolean formula | SNP | Allele | SNP | Allele | SNP | Allele |
| (A & B) \| (! C) | RAGE_8Kb_6006 | CC | FCGR3B_7.42Kb_5264 | AG | FCGR3B_7.42Kb_5137 | TT |
| (! A) \| (B & C) | FCGR3B_7.42Kb_5002 | AG | RAGE_8Kb_6006 | CC | FCGR3B_7.42Kb_5264 | AG |
| (! A) \| (B & C) | FCGR3B_7.42Kb_5167 | TT | RAGE_8Kb_6006 | CC | FCGR3B_7.42Kb_5264 | AG |
| (A & B) \| (! C) | RAGE_8Kb_5820 | AA | PAI1_10KB_2979 | TT | FCGR3B_7.42Kb_5137 | TT |

TABLE 5
Doberman

| | |
|---|---|
| A | B |

| Boolean formula | SNP | Allele | SNP | Allele |
|---|---|---|---|---|
| ((A \| (B & C)) \| D) \| E | FCGR2A_9.86Kb_8708 | GG | RAGE_8Kb_5847 | GG |

| | C | | D | | E | |
|---|---|---|---|---|---|---|
| Boolean formula | SNP | Allele | SNP | Allele | SNP | Allele |
| ((A \| (B & C)) \| D) \| E | RAGE_5KB_4329 | AA | RAGE_5KB_4766 | TT | RAGE_8Kb_6182 | TT |

TABLE 6
Golden Retriever

| | A | | B | | C | |
|---|---|---|---|---|---|---|
| Boolean formula | SNP | Allele | SNP | Allele | SNP | Allele |
| (! A & (B \| C)) | FCGR3B_7.42Kb_5239 | CC | RAGE_8Kb_5771 | CC | RAGE_8Kb_6182 | CC |
| \| (D & E & F) | | | | | | |
| | D | | E | | F | |
| | SNP | Allele | SNP | Allele | SNP | Allele |
| | RAGE_5KB_4805 | GG | FCGR3B_7.42Kb_4947 | CC | RAGE_8Kb_6006 | TT |

TABLE 7

Sequences around SNPs

| | | | |
|---|---|---|---|
| SNP Name | Before | Options | After |

| | | | |
|---|---|---|---|
| RAGE_8Kb_ | GGTGGTTCGGCAAAGTGCGCGC | AG | GAGCTTGGCGAGGTAGCAAATG |
| 6000 | GCGCAGCAGGCGGCGGGAAGGG | | CTATGGTCGCAGGGCTCAAAAT |
| | GCGGGCCAGGCCGAGAGTGCTC | | GGCTCCGGCCTCCTTCGGTGAC |
| | GGCTTTCTCTGGCCCCACCCCCT | | GTAGAGGGCAGAACTGGGGCTC |
| | CCGCCGCGGTCTTGTCGCCGTG | | GCCCCTCCCTTTCAGTGAAGAA |
| | GTGACTGCTCTACGATTGGCGG | | GGGAGCAGAACTGGCATCAGCT |
| | GCTTTGGGGTTCAAAGGCATCA | | CGACGTGTGGGTGGTGCGAGCA |
| | GCGCCGCCTCATCCGGGCCCTC | | TCGACCTGCGGCGCTGGTGTTA |
| | CGACTGCGGTCTCTCCGGGCTG | | ACCCATCTCCCTCGGCTGCGGG |
| | ATTGGCTAGTTTCTTGCAGCCCT | | ACGCAGGCCGCTCCTCCTTAGG |
| | GATTGGCCGAGATCGGAAAAGA | | TGACTTGCAGAACAGATCTCAA |
| | CGGCCG | | GGCCCAC |
| RAGE_8Kb_ | TGGTTCGGCAAAGTGCGCGCGC | ATG | GCTTGGCGAGGTAGCAAATGCT |
| 6002 | GCAGCAGGCGGCGGGAAGGGGC | | ATGGTCGCAGGGCTCAAAATGG |
| | GGGCCAGGCCGAGAGTGCTCGG | | CTCCGGCCTCCTTCGGTGACGT |
| | CTTTCTCTGGCCCCACCCCCTCC | | AGAGGGCAGAACTGGGGCTCGC |
| | GCCGCGGTCTTGTCGCCGTGGT | | CCCTCCCTTTCAGTGAAGAAGG |
| | GACTGCTCTACGATTGGCGGGC | | GAGCAGAACTGGCATCAGCTCG |
| | TTTGGGGTTCAAAGGCATCAGC | | ACGTGTGGGTGGTGCGAGCATC |
| | GCCGCCTCATCCGGGCCCTCCG | | GACCTGCGGCGCTGGTGTTAAC |
| | ACTGCGGTCTCTCCGOGCTGAT | | CCATCTCCCTCGGCTGCGGGAC |
| | TGGCTAGTTTCTTGCAGCCCTG | | GCAGCCCGCTCCTCCTTAGGTG |
| | ATTGGCCGAGATCGGAAAAGAC | | ACTTGCAGAACAGATCTCAAGG |
| | GGCCGGG | | CCCACAC |
| RAGE_8Kb_ | GAGCTCCGAAGCTCTGGATTGG | CT | TCTTGCAGCCCTGATTGGCCGA |
| 5959 | CTGGAACTCGACGGGAGATGGT | | GATCGGAAAAGACGGCCGGGAG |
| | GGTTCGGCAAAGTGCGCGCGCG | | CTTGGCGAGGTAGCAAATGCTA |
| | CAGCAGGCGGCGGGAAGGGGCG | | TGGTCGCAGGGCTCAAAATGGC |
| | GGCCAGGCCGAGAGTGCTCGGC | | TCCGGCCTCTTCGGTGACGTA |
| | TTTCTCTGGCCCCACCCCCTCCG | | GAGGGCAGAACTGGGGCTCGCC |
| | CCGCGGTCTTGTCGCCGTGGTG | | CCTCCCTTTCAGTGAAGAAGGG |
| | ACTGCTCTACGATTGGCGGGCT | | AGCAGAACTGGCATCAGCTCGA |
| | TTGGGGTTCAAAGGCATCAGCG | | CGTGTGGGTGGTGCGAGCATCG |
| | CCGCCTCATCCGGGCCCTCCGA | | ACCTGCGGCGCTGGTGTTAACC |
| | CTGCGGTCTCTCCGGGCTGATTG | | CATCTCCCTCGGCTGCGGGACG |
| | GCTAGT | | CAGCCCG |
| FCGR3B_ | TCCCTCCCAGGGCCCCATTTCTC | CT | GAGTTCTGGAGGAAAACAAGAG |
| 7.42Kb_5238 | ACCCCTGCCCCTCGCCTGGGCTT | | ACCTCTTCAGGGAATGCCCTTC |
| | CTCTTAACAGAAGGTGTGAATC | | TCCTTGGGTCTCATCCCCAGGGT |
| | TCATGGTCAAATTCATGCTAAG | | TGTGATATCTTCTTGTTCTTGGC |
| | AAGTGCGTCATAGACCTCAACA | | ACTGAGTGGATCCAAACCCGCC |
| | CACATGCTTTTTGAATTCTTGA | | AGGTAGGGAAACTTATTTTGAG |
| | AAAATAAGCTGGTGGAGACAAC | | ACTGATCAGATTTATCCATGTC |
| | ACTGCAGGGAGGGACCCTTCCT | | ACTAGATGCTTGCCTGTAGTCT |
| | CAGGGTCTGAAGATTTAAATGA | | CCTGTGGACCCGAGGGTGCTCA |
| | ATACAGGCAGGTAGGTCCAGGT | | TCCACCTCAGCTTCTCTTTCCGG |
| | GGATGGAGCCGGGTCTGGGCCA | | CTTTTTTCCTCCCCTTCCTGCCC |
| | GGCAGG | | CAT |
| PAI1_10KB_ | TCCCCTCAAAGACACACATCCA | CT | AGCACTGTCTCTGTGCCCATGA |
| 2979 | ATCTGAGGACTGAAGGGAACCA | | TGGCTCAGACCAACAAGTTCAA |
| | TCTAAAAGGATTAAAAAATATC | | CTACAGTAAGTTCAAGACTTCT |
| | TAGCCCCTTTACCCCCCAGCTC | | TCCAAAAGTCCCACAACTACTG |
| | ACCTGGCTTTCTCATAGGCATG | | CACCCCATCCCCTCGGGTATTCC |
| | ATTGGCAACTTACTGGGCAGAG | | CCTCTCAGGGAGGAGAAACCTT |
| | GGGCCGTGGACCAGCTGACGCG | | TGAATGTAGCCCAGCTTTGCCA |
| | TCTGATGCTGGTAAATGCCCTC | | GAGCCTCCCCAGGCAGGAGCTA |
| | TACTTCAACGGCCAGTGGAAGA | | CTGGGATGACAGGAGCAGCAGA |
| | CGCCCTTCCGGAGTCAGGCACC | | AAGTAGCTTCATCTCATGCAAG |
| | CACCACCGCCTCTTCCACAAAT | | CCAAAGCTGACATCCAGAAAGT |
| | CTGACGG | | CCCTCC |
| RAGE_8Kb_ | TCGGGAAAGTGCGCGCGCGCAG | CT | GGCGAGGTAGCAAATGCTATGG |
| 6006 | CAGGCGGCGGGAAGGGGCGGGC | | TCGCAGGGCTCAAAATGGCTCC |
| | CAGGCCGAGAGTGCTCGGCTTT | | GGCCTCCTTCGGTGACGTAGAG |
| | CTCTGGCCCCACCCCCTCCGCCG | | GGCAGAACTGGGGCTCGCCCCT |
| | CGGTCTTGTCGCCGTGGTGACT | | CCCTTTCAGTGAAGAAGGGAGC |
| | GCTCTACGATTGGCGGGCTTTG | | AGAACTGGCATCAGCTCGACGT |
| | GGGTTCAAAGGCATCAGCGCCG | | GTGGGTGGTGCGAGCATCGACC |
| | CCTCATCCGGGCCCTCCGACTG | | TGCGGCGCTGGTGTTAACCCAT |
| | CGGTCTCTCCGGGCTGATTGGCT | | CTCCCTCGGCTGCGGGACGCAG |
| | AGTTTCTTGCAGCCCTGATTGG | | CCCGCTCCTCCTTAGGTGACTTG |
| | CCGAGATCGGAAAAGACGGCCG | | CAGAACAGATCTCAAGGCCCAC |
| | GGAGC | | ACCTTT |
| FCGR3B_ | CCTGCCCCTCGCCTGGGCTTCTC | AG | CTTCAGGGAATGCCCTTCTCCTT |
| 7.42Kb_5264 | TTAACAGAAGGTGTGAATCTCA | | GGGTCTCATCCCCAGGGTTGTG |
| | TGGTCAAATTCATGCTAAGAAG | | ATATCTTCTTGTTCTTGGCACTG |
| | TGCGTCATAGACCTCAACACAC | | AGTGGATCCAAACCCGCCAGGT |
| | ATGCTTTTTGAATTCTTGAAAA | | AGGGAAACTTATTTTGAGACTG |
| | ATAAGCTGGTGGAGACAACACT | | ATCAGATTTATCCATGTCACTA |
| | GCAGGGAGGGACCCTTCCTCAG | | GATGCTTGCCTGTAGTCTCCTGT |
| | GGTCTGAAGATTTAAATGAATA | | GGACCCGAGGGTGCTCATCCAC |
| | CAGGCAGGTAGGTCCAGGTGGA | | CTCAGCTTCTCTTTCCGGCTTTT |
| | TGGAGCCGGGTCTGGGCCAGGC | | TTCCTCCCCTTCCTGCCCCATCC |
| | AGGAGAGTTCTGGAGGAAAACA | | TGGGGCTCACTTGTCAGAATTC |
| | AGAGACC | | AG |
| FCGR3B_ | TGATGGTGGGTTGAAGCTAAAG | TG | GACAACACTGCAGGGAGGGACC |
| 7.42Kb_5137 | AGCTCCTGTCCTCTCCTGCCCGC | | CTTCCTCAGGGTCTGAAGATTT |
| | TGTCCTTCCCTCTGCGCCTCCTT | | AAATGAATACAGGCAGGTAGGT |
| | CTCTGCCTTCCCTTCAACCAATT | | CCAGGTGGATGGAGCCGGGTCT |
| | AGTGACTTCCTCCCTCCCAGGG | | GGGCCAGGCAGGAGAGTTCTGG |
| | CCCCATTTCTCACCCCTGCCCCT | | AGGAAAACAAGAGACCTCTTCA |
| | CGCCTGGGCTTCTCTTAACAGA | | GGGAATGGCCTTCTCCTTGGGT |
| | AGGTGTGAATCTCATGGTCAAA | | CTCATCCCCAGGGTTGTGATAT |
| | TTCATGCTAAGAAGTGCGTCAT | | CTTCTTGTTCTTGGCACTGAGTG |
| | AGACCTCAACACACATGCTTTT | | GATCCAAACCCGCCAGGTAGGG |
| | TGAATTCTTGAAAAATAAGGTG | | AAACTTATTTTGAGACTGATCA |
| | GTGG | | GATTTA |
| FCGR3B_ | ACTTCCTGGCCACTGGACTTCC | AG | CATTTCTCACCCCTGCCCCTCGC |
| 7.42Kb_5002 | ACCTTTTCCAATAAGGCACCCC | | CTGGGCTTCTCTTAACAGAAGG |
| | GGAGCCAGGGCTACAGGCTCAC | | TGTGAATCTCATGGTCAAATTC |
| | AGACCAGCCCAGGCCAGTGGGT | | ATGCTAAGAAGTGCGTCATAGA |
| | CTCCGAGGGGCTGAGCTCACCT | | CCTCAACACACATGCTTTTTGA |
| | GGCTACTGTCACTGCTCAGCCC | | ATTCTTGAAAAATAAGCTGGTG |
| | TGGTGATGGTGGGTTGAAGCTA | | GAGACAACACTGCAGGGAGGGA |
| | AAGAGCTCCTGTCCTGTCCTGC | | CCCTTCCTCAGGGTCTGAAGAT |
| | CCGCTGTCCTTCCCTCTGCGCCT | | TTAAATGAATACAGGCAGGTAG |
| | CCTTCTCTGCCTTCCCTTCAACC | | GTCCAGGTGGATGGAGCCGGGT |
| | AATTAGTGACTTCCTCCCTCCC | | CTGGGCCAGGCAGGAGAGTTCT |
| | AGGGCC | | GGAGGA |
| FCGR3B_ | TCCTCTCCTGCCCGCTGTCCTTC | CT | GGGTCTGAAGATTTAAATGAAT |
| 7.42Kb_5167 | CCTCTGCGCCTCCTTCTCTGCCT | | ACAGGCAGGTAGGTCCAGGTGG |
| | TCCCTTCAACCAATTAGTGACT | | ATGGAGCCGGGTCTGGGCCAGG |
| | TCCTCCCTCCCAGGGCCCCATTT | | CAGGAGAGTTCTGGAGGAAAAC |
| | CTCACCCCTGCCCCTCGCCTGGG | | AAGAGACCTCTTCAGGGAATGC |
| | CTTCTCTTAACAGAAGGTGTGA | | CCTTCTCCTTGGGTCTCATCCCC |
| | ATCTCATGGTCAAATTCATGCT | | AGGGTTGTGATATCTTCTTGTTC |
| | AAGAAGTGCGTCATAGACCTCA | | TTGGCACTGAGTGGATCCAAAC |
| | ACACACATGCTTTTTGAATTCT | | CCGCCAGGTAGGGAAACTTATT |
| | TGAAAAATAAGCTGGTGGAGAC | | TTGAGACTGATCAGATTTATCC |
| | AACACTGCAGGGAGGGACGCTT | | ATGTCACTAGATGCTTGCCTGT |
| | CCTC | | AGTCT |
| RAGE_8Kb_ | TCCGAGTAGCTGCCAGTCAGGG | AT | TCTCTGGCCCCACCCCCTCCGCC |
| 5820 | CCAAGGGCCAGAAGCAATTGGT | | GCGGTCTTGTCGCCGTGGTGAC |
| | CCGGGACCACACAGGCCTCGCC | | TGCTCTACGATTGGCGGGCTTT |
| | TCCTCCGAGCCCTTTCTTTGCTT | | GGGGTTCAAAGGCATCAGCGCC |
| | CACTTCCCCTTTCCGAGAACGT | | GCCTCATCCGGGCCCTCCGACT |
| | CCGGATTCCTATTGGACTTTGG | | GCGGTCTCTCCGGGCTGATTGG |
| | AGCGTAGAGCTCCGAAGCTCTG | | CTAGTTTCTTGCAGCCCTGATTG |
| | GATTGGCTGGAACTCGACGGGA | | GCCGAGATCGGAAAAGACGGCC |
| | GATGGTGGTTCGGCAAAGTGCG | | GGGAGCTTGGCGAGGTAGCAAA |
| | CGCGCGCAGCAGGCGGCGGGAA | | TGCTATGGTCGCAGGGCTCAAA |
| | GGGGCGGGCCAGGCCGAGAGTG | | ATGGCTCCGGCCTCCTTCGGTG |
| | CTCGGCT | | ACGTA |
| FCGR2A_ | TCCTTTCTCTTTCCCCTCCTCTC | AG | GGGCCTTGTTCTCTGAACAGAA |
| 9.86Kb_8708 | AGAGAAGCAGAGGATAGGCAG | | ATAGGAAGAGATTGATTGATTG |
| | CCATGGTGCACAGGTGCTTTAA | | ATTGCACCTCGGTGAAGTACAT |
| | CCCTTCTGGTTCTGAGAGGGTG | | GCTGCTGCGCACTCCTTACTCA |
| | AGACATCACAGATATTGTCCCA | | ACACTAGGAATCTCCCACCTCC |
| | GAAAATAACTCACCCCCCTCTC | | CAGGCTCCCAGGGAGGGGATGG |
| | TCAGTAAAATCAAGAGCCCAAA | | GGGTGCAGTTCTCCCTGGGGCA |
| | CATTTTTCGTCTTAGCTGCACGC | | CTGACCCCAGGGCTCCTTAGAC |
| | GAAATCCCATCATCTGCCTAGA | | TAGACCTCCAGCCTTTCTTTCTT |
| | TTATCTCCATGTGTTGATAAAT | | TTTCTTTCTGAGGCCACAGAGA |
| | CCTCCACTTTGCATGACTCTGA | | CCCCTCTGTACTTTGGTGCCAA |
| | GGGCTTC | | GACAGG |
| RAGE_8Kb_ | GGCCAGAAGCAATTGGTCCGGG | CTG | TCTTGTCGCCGTGGTGACTGCTC |
| 5847 | ACCACACAGGCCTCGCCTCCTC | | TACGATTGGCGGGCTTTGGGGT |
| | CGAGCCCTTCTTTGCTTCACTT | | TCAAAGGCATCAGCGCCGCCTC |
| | CCCCTTTCCGAGAACGTCCGGA | | ATCCGGGCCCTCCGACTGCGGT |
| | TTCCTATTGGACTTTGGAGCGT | | CTCTCCGGGCTGATTGGCTAGT |
| | AGAGCTCCGAAGCTCTGGATTG | | TTCTTGCAGCCCTGATTGGCCG |
| | GCTGGAACTCGACGGGAGATGG | | AGATCGGAAAAGACGGCCGGGA |
| | TGGTTCGGCAAAGTGCGCGCGC | | GCTTGGCGAGGTAGCAAATGCT |
| | GCAGCAGGCGGCGGGAAGGGGC | | ATGGTCGCAGGGCTCAAAATGG |
| | GGGCCAGGCCGAGAGTGCTCGG | | CTCCGGCCTCCTTCGGTGACGT |
| | CTTTCTCTGGCCCCACCCCCTCC | | AGAGGGCAGAACTGGGGCTCGC |
| | GCCGCG | | CCCTCC |
| RAGE_5KB_ | CTTGGGCAGGGCTGGATTCAGT | AT | AAAAGCCAGGTGTGGGGGAAAG |
| 4329 | AATTTTGAGGAAGCGCCACCTT | | TCAAATCACCAGTGTCCCATCC |
| | CCCCTGTGAGTGACACATCTTT | | TTGGCCAGAACCCTACCATCTG |
| | AAGTCTTCTTTTTAACCTATTTG | | AGTCCCTCAAACATCCTCAGGA |
| | CAGATTGGAGAGGGAAGAACA | | TTTTATAAGACTGTCATAGTGG |
| | GGGAGGGGGTTATTGCCAAATA | | GGAACCTCTCCTGTCAAAGACC |
| | TGTTAAATGTGGGTTGGGGTGC | | AGGCAGGACTGGAGGGGAGCAG |
| | TTGTGTATGTATCTCCCTCAATT | | GTTAGATGGGTGATGGGTGGAG |
| | TCCCCAGAAACGAGGCATTCTT | | GGTGGGAGGCACGGGCCGGGGG |
| | TTTTTCTCAGTCTAAAATCAAG | | CAGTTCTCTCCTCACTTGTAAA |
| | AGGGTGGGGGGAGAGAGGAGG | | CTTGTAGTTTCACAGAAAAGGA |
| | CATGTCAT | | AAAAAAA |
| RAGE_5KB_ | ACGGGCCGGGGGCAGTTCTCTC | TG | CAAGGCCAGGCCAGGCCTGAAC |
| 4766 | CTCACTTGTAAACTTGTAGTTT | | CCTGTTGCCCAGCAACCTTACC |
| | CACAGAAAAGGAAAAAAAAAA | | TAAGCAACATGGGGCTCCCATC |
| | TGCAGTTTTAAATAAAGAAATT | | GTCCACCAGGCAAGCCCTCAGT |
| | TCTTTTTTCCCTGGGTTTAGTTG | | GGACTGATGGAATGGGTTAGGG |
| | AGCATTATTTTCAAAAACATGA | | GTCCTGAATACTAAGAAACCTT |
| | TAAACCCCAGAATAAAATTCTT | | AGGAGGTCCACTCCCAACCCCC |
| | TCATAAAACCCCAAACGGTGTT | | ACGGACATGGCTGTGCCCAGAC |
| | TTCCCTTCCAGCTACCCACTCCC | | TGGCACTGCCTAAGGGTGGGGT |
| | AACCTTACCCTCACCACCCAGG | | GATCATTGTTTCTCCTAGTACCT |
| | AGCACCCATGGTTCACCCTCAA | | GAAGGACTCTTGTCTAAGAAGC |
| | CCCTCCC | | ATGAAT |
| RAGE_8Kb_ | GCGGTCTCTCCGGGCTGATTGG | CT | CTCCCTCGGCTGCGGGACGCAG |
| 6182 | CTAGTTTCTTGCAGCCCTGATTG | | CCCGCTCCTCCTTAGGTGACTTG |
| | GCCGAGATCGGAAAAGACGGCC | | CAGAACAGATCTCAAGGCCCAC |
| | GGGAGCTTGGCGAGGTAGCAAA | | ACCTTTCTAACGTTGACACAGG |
| | TGCTATGGTCGCAGGGCTCAAA | | ATGACAGAGTTGACCCCGGCCC |
| | ATGGCTCCGGCCTCCTTCGGTG | | CGTTTTAAACCTGAAAAGCGAA |
| | ACGTAGAGGGCAGAACTGGGGC | | CTAGCTCCACCCCTTCGTGAGT |
| | TCGCCCCTCCCTTTCAGTGAAG | | AGGTGCCGAGGGGGCAAGGGCC |
| | AAGGGAGCAGAACTGGCATCAG | | GCCCTCCTGAGCGACCCGCGGC |
| | CTCGACGTGTGGGTGGTGCGAG | | GGAATGGGGTTAGGCCCGCCCC |
| | CATCGACCTGCGGCGCTGGTGT | | TTCCGTCCTGTAGTGTGTCCCGC |
| | TAACCCA | | AGAAG |
| FCGR3B_ | CCCTCCCAGGGCCCCATTTCTCA | CT | AGTTCTGGAGGAAAACAAGAGA |
| 7.42Kb_5239 | CCCCTGCCCCTCGCCTGGGCTTC | | CCTCTTCAGCGAATGCCCTTCTC |
| | TCTTAACAGAAGGTGTGAATCT | | CTTGGGTCTCATCCCCAGGGTT |
| | CATGGTCAAATTCATGCTAAGA | | GTGATATCTTCTTGTTTCTTGGCA |
| | AGTGCGTCATAGACCTCAACAC | | CTGAGTGGATCCAAACCCGCCA |
| | ACATGCTTTTTGAATTCTTGAA | | GGTAGGGAAACTTATTTTGAGA |
| | AAATAAGCTGGTGGAGACAACA | | CTGATCAGATTTATCCATGTCA |
| | CTGCAGGGAGGGACCCTTCCTC | | CTAGATGCTTGCCTGTAGTCTCC |
| | AGGGTCTGAAGATTTAAATGAA | | TGTGGACCCGAGGGTGCTCATC |
| | TACAGGCAGGTAGGTCCAGGTG | | CACCTCAGCTTCTCTTTCCGGCT |
| | GATGGAGCCGGGTCTGGGCCAG | | TTTTTCCTCCCCTTCCTGCCCCA |
| | GCAGGA | | TC |
| RAGE_8Kb_ | CATGCGACAGAATTGGTGTCCG | CT | GCGCAGCAGGCGGCGGGAAGGG |
| 5771 | TTGGACCTGGTCGGGGAGCTTG | | GCGGGCCAGGCCGAGAGTGCTC |
| | ATTCGTCCGAGTAGCTGCCAGT | | GGCTTTCTCTGGCCCCACCCCCT |
| | CAGGGCCAAGGGCCAGAAGCAA | | CCGCCGCGGTCTTGTCGCCGTG |
| | TTGGTCCGGGACCACACAGGCC | | GTGACTGCTCTACGATTGGCGG |
| | TCGCCTCCTCCGAGCCCTTTCTT | | GCTTTGGGGTTCAAAGGCATCA |
| | TGCTTCACTTCCCCTTTCCGAGA | | GCGCCGCCTCATCCGGGCCCTC |
| | ACGTCCGGATTCCTATTGGACT | | CGACTGCGGTCTCTCCGGGCTG |
| | TTGGAGCGTAGAGCTCCGAAGC | | ATTGGCTAGTTTCTTGCAGCCCT |
| | TCTGGATTGGCTGGAACTCGAC | | GATTGGCCGAGATCGGAAAAGA |
| | GGGAGATGGTGGTTCGGCAAAG | | CGGCCGGGAGCTTGGCGAGGTA |
| | TGCGCG | | GCAAA |
| RAGE_5KB_ | AGTTTCACAGAAAAGGAAAAA | TG | TTACCTAAGCAACATGGGGCTC |
| 4805 | AAAAATGCAGTTTTAAATAAAG | | CCATCGTCCACCAGGCAAGCCC |
| | AAATTTCTTTTTTCCCTGGGTTT | | TCAGTGGACTGATGGAATGGGT |
| | AGTTGAGCATTATTTTCAAAAA | | TAGGGGTCCTGAATACTAAGAA |
| | CATGATAAACCCCAGAATAAAA | | ACCTTAGGAGGTCCACTCCCAA |
| | TTCTTTCATAAAACCCCAAACG | | CCCCCACGGACATGGCTGTGCC |
| | GTGTTTCCCTTCCAGCTACCCA | | CAGACTGGCACTGCCTAAGGGT |
| | CTCCCAACCTTACCCTCACCAC | | GGGGTGATCATTGTTTCTCCTA |
| | CCAGGAGCACCCATGGTTCACC | | GTACCTGAAGGACTCTTGTCTA |
| | CTCAACCCTCCCCCAAGGCCAG | | AGAAGCATGAATTCCTAGCATT |
| | GCCAGGCCTGAACCCTGTTGCC | | CCCCGTGGCCGGATAGGACAGG |
| | CAGCAAC | | ATGGAAA |
| FCGR3B_ | GCCGTGTGTTGGGGGGATGCGG | CT | CCTCCTTCTCTGCCTTCCCTTCA |
| 7.42Kb_4947 | CTAGGGAGAGTAGAACAGGGTA | | ACCAATTAGTGACTTCCTCCCT |
| | GCAATCTTAAGACTTCCTGGCC | | CCCAGGGCCCCATTTCTCACCC |
| | ACTGCACTTCCACCTTTTCCAAT | | CTGCCCCTCGCCTGGGCTTCTCT |
| | AAGGCACCCCCGAGCCAGGGCT | | TAACAGAAGGTGTGAATCTCAT |
| | ACAGGCTCACAGACCAGCCCAG | | GGTCAAATTCATGCTAAGAAGT |
| | GCCAGTGGGTCTCCGAGGGGCT | | GCGTCATAGACCTCAACACACA |
| | GAGCTCACCTGGCTACTGTCAC | | TGCTTTTTGAACTTCTTGAAAAA |
| | TGCTCAGCCCTGGTGATGGTGG | | TAAGCTGGTGGAGACAACACTG |
| | GTTGAAGCTAAAGAGCTCCTGT | | CAGGGAGGGACCCTTCCTCAGG |
| | CCTCTCCTGCCCGCTGTCCTTCC | | GTCTGAAGATTTAAATGAATAC |
| | CTCTGC | | AGGCA |

1. A method for assessing a nutritional requirement, disease susceptibility or behavioral characteristic of a dog, the method comprising:
(a) determining the nucleotide present at one or more SNP positions in the dog's genome;
(b) identifying therefrom the genetic breed inheritance of the dog;
(c) thereby determining a nutritional requirement, disease susceptibility or behavioral characteristic of the dog.

2. A method according to claim 1, wherein the genetic breed inheritance of the dog is identified from a combination of the nucleotides present at two or more SNP positions.

3. A method according to claim 1, wherein at least 10 different SNP positions are typed.

4. A method according to claim 1, wherein the genetic breed inheritance is the dog's breed.

5. A method according to claim 1, wherein the one or more SNP positions are any of those identified in SEQ ID NO:s 1 or 4 to 23.

6. A method according to claim 1, wherein the nucleotide present at one or more breed-specific SNP positions is detected by contacting a polynucleotide or protein from the dog with a specific binding agent and determining whether the agent binds to the polynucleotide or protein.

7. A method according to claim 6, wherein the agent is a polynucleotide.

8. A method according to claim 1, wherein the nucleotide present at one or more breed-specific SNP positions is detected by measuring the mobility of a polynucleotide or protein of the dog during electrophoresis.

9. A method according to claim 1, wherein the nucleotide present at one or more SNP positions in the dog is used to distinguish between the following breeds: Labrador retriever, Golden retriever, German Shepherd, Dachshund, Shih Tzu, Yorkshire terrier, Poodle, Rottweiler, Boxer and Cocker spaniel.

10. A method according to claim 1, wherein the dog has the physical features of a mongrel and/or is suspected of being a mongrel and/or is suspected of having a nutritional, medical or behavioral problem.

11. A method of determining the genetic breed background of a dog, the method comprising:
(a) determining the nucleotide present at one or more SNP positions in the dog; and
(b) identifying therefrom the genetic breed inheritance of the dog.

12. An isolated polynucleotide that comprises a sequence of any one of SEQ ID NO:s 1 or 4 to 23 or a polypeptide encoded thereof.

13. A probe, primer or antibody which is capable of detecting a polynucleotide or polypeptide according to claim 12.

14. A kit for carrying out the method of claim 1, comprising means for detecting the nucleotide present at one or more breed-specific SNP positions.

15. A method of identifying one or more SNP positions which can be used to determine the breed inheritance of a dog, the method comprising:
(a) screening the nuclear genome, RNA or proteins of dogs from one or defined breeds;
(b) identifying one or more SNP positions in the nuclear genome, RNA or proteins; and
(c) determining the relationship between the nucleotide present at one or more SNP positions and one or more dog breeds.

16. A method of preparing customized food for a dog, comprising:
(a) determining one or more nutritional requirements of the dog by a method according to claim 1;
(b) generating a customized dog food formulation that corresponds to the nutritional requirements of the dog; and
(c) preparing a dog food according to the customized dog food formulation.

17. A method according to claim 16, wherein the customized dog food comprises components suitable for the breed(s) which contributed to the genetic breed inheritance of the dog, and which does not comprise components that are not suitable for the breed(s) which contributed to the genetic breed inheritance of the dog.

18. A method according to claim 17, wherein the food does not comprise ingredients which are poorly tolerated, cause allergies, are abnormally processed or stored, or contribute to diseases or conditions typically suffered by the breed(s) which have contributed to the genetic breed inheritance of the dog or wherein the food contains ingredients which have nutritional or medical benefits for the breed(s) which have contributed to the genetic breed inheritance of the dog.

19. A method according to claim 16, wherein the food contains: cocoa flavanols, other plant flavanols, lycopene, curcumin, minerals, trace metals, Echineacea, phosphatidyl serine, L-arginine, ginseng, psyllium, prebiotics, probiotics, phyto-oestrogens, phyto-chemicals, soluble fiber, PUFAs or phospholipids; and/or does not contain or has low levels of: gluten-containing grains such as wheat, animal proteins, milk, eggs, soy, peanuts, shellfish, fruits, tree nuts, copper, saturated fats or salt.

20. A method according to claim 16, further comprising providing the dog's owner, the person responsible for feeding the dog or a vet with the customized food and/or providing the customized food to the dog.

21. A method of providing food customized to the nutritional requirements of a dog, the method comprising providing to:
(a) the dog's owner, the person responsible for feeding the dog or a vet; or
(b) to the dog;
a food which contains components suitable for the breed(s) which have contributed to the genetic breed inheritance of the dog, and which does not contain components that are not suitable for the breed(s) which have contributed to the genetic breed inheritance of the dog, wherein the breed inheritance of the dog has been identified by determining the nucleotide present at one or more breed-specific SNP positions in the dog genome.

22. A labeled dog food product, wherein the food product is customized for one or more breeds and the label provides an indication of one or more breed specific genomic SNPs present in said breed(s).

23. A method of treating a dog for a disease that occurs in a dog breed, the method comprising administering to the dog an effective amount of a therapeutic compound which prevents or treats the disease, wherein the dog has been identified as being susceptible to that disease by a method according to claim 1.

24. A database comprising information relating to breed-specific genomic SNPs and optionally the nutritional, medical or behavioral needs of said breeds.

25. A method for determining a nutritional requirement, disease susceptibility or behavioral characteristic of a dog, the method comprising:
(i) inputting data of the nucleotide present at one or more breed-specific SNP positions in the dog to a computer system;
(ii) comparing the data to a computer database, which database comprises information relating to breed-specific SNPs and the nutritional requirements, disease susceptibility or behavioral characteristics of the breeds; and
(iii) determining on the basis of the comparison a nutritional requirement, disease susceptibility or behavioral characteristic of the dog.

26. A method for identifying the genetic breed inheritance of a dog, the method comprising:
(i) inputting genetic data from the dog to a computer system;
(ii) comparing the data to a computer database, which database comprises information relating to breed-specific genomic SNPs; and
(iii) determining on the basis of the comparison the nucleotide present at one or more breed-specific SNP positions, thereby identifying the breed inheritance of the dog.

27. A method according to claim 25, wherein the one or more SNP positions are any of those identified in SEQ ID NO:s 1 or 4 to 23.

28. A method according to claim 26, wherein the one or more SNP positions are any of those identified in SEQ ID NO:s 1 or 4 to 23.

29. A computer program comprising program code that, when executed on a computer system, instructs the computer system to perform all the steps of claim 25.

30. A computer program comprising program code that, when executed on a computer system, instructs the computer system to perform all the steps of claim 26.

31. A computer system arranged to perform a method according to claim 25 comprising:
(i) means for receiving data of the nucleotide present at one or more breed-specific genomic SNP positions in the dog;
(ii) a module for comparing the data with a database comprising information relating to breed-specific genomic SNPs and the nutritional requirements, disease susceptibility or behavioral characteristics of the breeds; and
(iii) means for determining on the basis of said comparison a nutritional requirement, disease susceptibility or behavioral characteristic of the dog.

32. A computer system arranged to perform a method according to claim 26 comprising:
(i) means for receiving genetic data from the dog;
(ii) a module for comparing the data with a database comprising information relating to breed-specific genomic SNPs; and
(iii) means for determining on the basis of said comparison the breed inheritance of the dog.

33. A method according to claim 25, further comprising:
(iv) electronically processing the nutritional requirement information to generate a customized dog food formulation;
(v) generating electronic manufacturing instructions to control the operation of food manufacturing apparatus in accordance with the customized dog food formulation; and
(vi) manufacturing the customized dog food according to the electronic manufacturing instructions.

34. A computer system according to claim 31, further comprising:
(iv) means for processing the nutritional requirement information to generate a customized dog food formulation;
(v) means for generating electronic manufacturing instructions to control the operation of food manufacturing apparatus in accordance with the customized dog food formulation; and
(vi) a food product manufacturing apparatus.

35. A method of determining the degree of relatedness between two dogs of the same breed, the method comprising comparing the genetic breed inheritance of a dog with the genetic breed inheritance of another dog of the same breed, and determining from the comparison the degree of relatedness between the two dogs.

36. A method of selecting one or more dogs for breeding with a subject dog, the method comprising:
(a) comparing the genetic breed inheritance of the subject dog with the genetic breed inheritance of each dog in a test group of two or more dogs of the same breed and of the opposite sex to the subject dog;
(b) determining from the comparison the degree of relatedness between the subject dog and each dog in the test group; and
(c) selecting one or more dogs from the test group for breeding with the subject dog.

37. A method according to claim 36, wherein the selection is further based on the geographical location, age, breeding status, medical history, disease susceptibility or a physical characteristic of the dogs in the test group.

38. A method according to claim 36, wherein the test group consists of at least 10 dogs.

39. A method according to claim 36, wherein at least 5 dogs in the test group are selected for breeding with the subject dog.

40. A method of providing a recommendation of one or more dogs for breeding with a subject dog, wherein the one or more dogs are selected by a method according to claim 36.

41. A method of breeding dogs, wherein a subject dog is bred with a dog selected by a method according to claim 36.

42. A database comprising information relating to the genetic breed background and sex of one or more dogs of the same breed and optionally the breeding status, age, geographical location, medical history, disease susceptibility or a physical characteristic of said dogs.

43. A method of selecting one or more dogs for breeding with a subject dog, the method comprising:
(i) inputting data relating to the genetic breed inheritance of a subject dog to a computer system;
(ii) comparing the data to a computer database, which database comprises information relating to the genetic breed background and sex of each dog in a test group of two or more dogs of the same breed;
(iii) determining on the basis of the comparison the degree of relatedness between the subject dog and each dog in the test group; and
(iv) selecting one or more dogs from the test group for breeding with the subject dog.

44. A computer program comprising program code that, when executed on a computer system, instructs the computer system to perform all the steps of claim 43 when said program is run on a computer.

45. A computer system arranged to perform a method according to claim 43 comprising:
(i) means for receiving data of the genetic breed inheritance of a subject dog;
(ii) a module for comparing the data with a database comprising information relating to the genetic breed background and sex of each dog in a test group of two or more dogs of the same breed;
(iii) means for determining on the basis of said comparison the degree of relatedness between the subject dog and each dog in the test group; and
(iv) means for selecting one or more dogs from the test group for breeding with the subject dog.


SEQUENCE LISTING (23 sequences)

SEQ ID NO: 1 - DNA, 361 bp, Canis familiaris
  misc_feature (116)..(116): n is a single nucleotide polymorphism and may be c or t
actccacttcacctccagcaaaacagagcataacttggaagaaacatctgatcagaaagatagcctaatatgggagaagaaaaacatgaccacatagttcctgtggttaccagccnagcccttggctcattgctggagttataaaacccaagaccagaaaatagaagcagcatctgcccagggcagcctcactgagaagatgcattgtcttcctctcaccctgctgctccttctcctatgttccagagcagaagctggtgagtcttgggatccttccccctggaaacggcaggatcagcaccccaaaaccaagtttagtctgaatatagctgactcataagcaaggtggcaggatctctct

SEQ ID NO: 2 - DNA, 19 bp, Artificial sequence
  Primer
actccacttcacctccagc

SEQ ID NO: 3 - DNA, 19 bp, Artificial sequence
  Primer
agagatcctgccaccttgc

SEQ ID NO: 4 - DNA, 500 bp, Canis familiaris
  misc_feature (251)..(251): n is a single nucleotide polymorphism and may be a or g
ggtggttcggcaaagtgcgcgcgcgcagcaggcggcgggaaggggcgggccaggccgagagtgctcggctttctctggccccaccccctccgccgcggtcttgtcgccgtggtgactgctctacgattggcgggctttggggttcaaaggcatcagcgccgcctcatccgggccctccgactgcggtctctccgggctgattggctagtttcttgcagccctgattggccgagatcggaaaagacggccgngagcttggcgaggtagcaaatgctatggtcgcagggctcaaaatggctccggcctccttcggtgacgtagagggcagaactggggctcgcccctccctttcagtgaagaagggagcagaactggcatcagctcgacgtgtgggtggtgcgagcatcgacctgcggcgctggtgttaacccatctccctcggctgcgggacgcagcccgctcctccttaggtgacttgcagaacagatctcaaggcccac

SEQ ID NO: 5 - DNA, 500 bp, Canis familiaris
  misc_feature (251)..(251): n is a single nucleotide polymorphism and may be a, t or g
tggttcggcaaagtgcgcgcgcgcagcaggcggcgggaaggggcgggccaggccgagagtgctcggctttctctggccccaccccctccgccgcggtcttgtcgccgtggtgactgctctacgattggcgggctttggggttcaaaggcatcagcgccgcctcatccgggccctccgactgcggtctctccgggctgattggctagtttcttgcagccctgattggccgagatcggaaaagacggccgggngcttggcgaggtagcaaatgctatggtcgcagggctcaaaatggctccggcctccttcggtgacgtagagggcagaactggggctcgcccctccctttcagtgaagaagggagcagaactggcatcagctcgacgtgtgggtggtgcgagcatcgacctgcggcgctggtgttaacccatctccctcggctgcgggacgcagcccgctcctccttaggtgacttgcagaacagatctcaaggcccacac

SEQ ID NO: 6 - DNA, 500 bp, Canis familiaris
  misc_feature (251)..(251): n is a single nucleotide polymorphism and may be c or t
gagctccgaagctctggattggctggaactcgacgggagatggtggttcggcaaagtgcgcgcgcgcagcaggcggcgggaaggggcgggccaggccgagagtgctcggctttctctggccccaccccctccgccgcggtcttgtcgccgtggtgactgctctacgattggcgggctttggggttcaaaggcatcagcgccgcctcatccgggccctccgactgcggtctctccgggctgattggctagtntcttgcagccctgattggccgagatcggaaaagacggccgggagcttggcgaggtagcaaatgctatggtcgcagggctcaaaatggctccggcctccttcggtgacgtagagggcagaactggggctcgcccctccctttcagtgaagaagggagcagaactggcatcagctcgacgtgtgggtggtgcgagcatcgacctgcggcgctggtgttaacccatctccctcggctgcgggacgcagcccg

SEQ ID NO: 7 - DNA, 500 bp, Canis familiaris
  misc_feature (251)..(251): n is a single nucleotide polymorphism and may be c or t
tccctcccagggccccatttctcacccctgcccctcgcctgggcttctcttaacagaaggtgtgaatctcatggtcaaattcatgctaagaagtgcgtcatagacctcaacacacatgctttttgaattcttgaaaaataagctggtggagacaacactgcagggagggacccttcctcagggtctgaagatttaaatgaatacaggcaggtaggtccaggtggatggagccgggtctgggccaggcaggngagttctggaggaaaacaagagacctcttcagggaatgcccttctccttgggtctcatccccagggttgtgatatcttcttgttcttggcactgagtggatccaaacccgccaggtagggaaacttattttgagactgatcagatttatccatgtcactagatgcttgcctgtagtctcctgtggacccgagggtgctcatccacctcagcttctctttccggcttttttcctccccttcctgccccat

SEQ ID NO: 8 - DNA, 500 bp, Canis familiaris
  misc_feature (251)..(251): n is a single nucleotide polymorphism and may be c or t
tcccctcaaagacacacatccaatctgaggactgaagggaaccatctaaaaggattaaaaaatatctagcccctttaccccccagctcacctggctttctcataggcatgattggcaacttactgggcagaggggccgtggaccagctgacgcgtctgatgctggtaaatgccctctacttcaacggccagtggaagacgcccttcccggagtcaggcacccaccaccgcctcttccacaaatctgacggnagcactgtctctgtgcccatgatggctcagaccaacaagttcaactacagtaagttcaagacttcttccaaaagtcccacaactactgcaccccatcccctcgggtattcccctctcagggaggagaaacctttgaatgtagcccagctttgccagagcctccccaggcaggagctactgggatgacaggagcagcagaaagtagcttcatctcatgcaagccaaagctgacatccagaaagtccctcc

SEQ ID NO: 9 - DNA, 500 bp, Canis familiaris
  misc_feature (251)..(251): n is a single nucleotide polymorphism and may be c or t
tcggcaaagtgcgcgcgcgcagcaggcggcgggaaggggcgggccaggccgagagtgctcggctttctctggccccaccccctccgccgcggtcttgtcgccgtggtgactgctctacgattggcgggctttggggttcaaaggcatcagcgccgcctcatccgggccctccgactgcggtctctccgggctgattggctagtttcttgcagccctgattggccgagatcggaaaagacggccgggagctnggcgaggtagcaaatgctatggtcgcagggctcaaaatggctccggcctccttcggtgacgtagagggcagaactggggctcgcccctccctttcagtgaagaagggagcagaactggcatcagctcgacgtgtgggtggtgcgagcatcgacctgcggcgctggtgttaacccatctccctcggctgcgggacgcagcccgctcctccttaggtgacttgcagaacagatctcaaggcccacaccttt

SEQ ID NO: 10 - DNA, 500 bp, Canis familiaris
  misc_feature (251)..(251): n is a single nucleotide polymorphism and may be a or g
cctgcccctcgcctgggcttctcttaacagaaggtgtgaatctcatggtcaaattcatgctaagaagtgcgtcatagacctcaacacacatgctttttgaattcttgaaaaataagctggtggagacaacactgcagggagggacccttcctcagggtctgaagatttaaatgaatacaggcaggtaggtccaggtggatggagccgggtctgggccaggcaggagagttctggaggaaaacaagagaccncttcagggaatgcccttctccttgggtctcatccccagggttgtgatatcttcttgttcttggcactgagtggatccaaacccgccaggtagggaaacttattttgagactgatcagatttatccatgtcactagatgcttgcctgtagtctcctgtggacccgagggtgctcatccacctcagcttctctttccggcttttttcctccccttcctgccccatcctggggctcacttgtcagaattcag

SEQ ID NO: 11 - DNA, 500 bp, Canis familiaris
  misc_feature (251)..(251): n is a single nucleotide polymorphism and may be t or g
tgatggtgggttgaagctaaagagctcctgtcctctcctgcccgctgtccttccctctgcgcctccttctctgccttcccttcaaccaattagtgacttcctccctcccagggccccatttctcacccctgcccctcgcctgggcttctcttaacagaaggtgtgaatctcatggtcaaattcatgctaagaagtgcgtcatagacctcaacacacatgctttttgaattcttgaaaaataagctggtggngacaacactgcagggagggacccttcctcagggtctgaagatttaaatgaatacaggcaggtaggtccaggtggatggagccgggtctgggccaggcaggagagttctggaggaaaacaagagacctcttcagggaatgcccttctccttgggtctcatccccagggttgtgatatcttcttgttcttggcactgagtggatccaaacccgccaggtagggaaacttattttgagactgatcagattta

SEQ ID NO: 12 - DNA, 500 bp, Canis familiaris
  misc_feature (251)..(251): n is a single nucleotide polymorphism and may be a or g
acttcctggccactgcacttccaccttttccaataaggcaccccggagccagggctacaggctcacagaccagcccaggccagtgggtctccgaggggctgagctcacctggctactgtcactgctcagccctggtgatggtgggttgaagctaaagagctcctgtcctctcctgcccgctgtccttccctctgcgcctccttctctgccttcccttcaaccaattagtgacttcctccctcccagggccncatttctcacccctgcccctcgcctgggcttctcttaacagaaggtgtgaatctcatggtcaaattcatgctaagaagtgcgtcatagacctcaacacacatgctttttgaattcttgaaaaataagctggtggagacaacactgcagggagggacccttcctcagggtctgaagatttaaatgaatacaggcaggtaggtccaggtggatggagccgggtctgggccaggcaggagagttctggagga

SEQ ID NO: 13 - DNA, 500 bp, Canis familiaris
  misc_feature (251)..(251): n is a single nucleotide polymorphism and may be c or t
tcctctcctgcccgctgtccttccctctgcgcctccttctctgccttcccttcaaccaattagtgacttcctccctcccagggccccatttctcacccctgcccctcgcctgggcttctcttaacagaaggtgtgaatctcatggtcaaattcatgctaagaagtgcgtcatagacctcaacacacatgctttttgaattcttgaaaaataagctggtggagacaacactgcagggagggacccttcctcngggtctgaagatttaaatgaatacaggcaggtaggtccaggtggatggagccgggtctgggccaggcaggagagttctggaggaaaacaagagacctcttcagggaatgcccttctccttgggtctcatccccagggttgtgatatcttcttgttcttggcactgagtggatccaaacccgccaggtagggaaacttattttgagactgatcagatttatccatgtcactagatgcttgcctgtagtct

SEQ ID NO: 14 - DNA, 500 bp, Canis familiaris
  misc_feature (251)..(251): n is a single nucleotide polymorphism and may be a or t
tccgagtagctgccagtcagggccaagggccagaagcaattggtccgggaccacacaggcctcgcctcctccgagccctttctttgcttcacttcccctttccgagaacgtccggattcctattggactttggagcgtagagctccgaagctctggattggctggaactcgacgggagatggtggttcggcaaagtgcgcgcgcgcagcaggcggcgggaaggggcgggccaggccgagagtgctcggctntctctggccccaccccctccgccgcggtcttgtcgccgtggtgactgctctacgattggcgggctttggggttcaaaggcatcagcgccgcctcatccgggccctccgactgcggtctctccgggctgattggctagtttcttgcagccctgattggccgagatcggaaaagacggccgggagcttggcgaggtagcaaatgctatggtcgcagggctcaaaatggctccggcctccttcggtgacgta

SEQ ID NO: 15 - DNA, 500 bp, Canis familiaris
  misc_feature (251)..(251): n is a single nucleotide polymorphism and may be a or g
tcctttctctttcccctcctctcagagaagcagaggataggcagccatggtgcacaggtgctttaacccttctggttctgagagggtgagacatcacagatattgtcccagaaaataactcacccccctctctcagtaaaatcaagagcccaaacatttttcgtcttagctgcacgcgaaatcccatcatctgcctagattatctccatgtgttgataaatcctccactttgcatgactctgagggcttcngggccttgttctctgaacagaaataggaagagattgattgattgattgcacctcggtgaagtacatgctgctgcgcactccttactcaacactaggaatctcccacctcccaggctcccagggaggggatgggggtgcagttctccctggggcactgaccccagggctccttagactagacctccagcctttctttctttttctttctgaggccacagagacccctctgtactttggtgccaagacagg

SEQ ID NO: 16 - DNA, 500 bp, Canis familiaris
  misc_feature (251)..(251): n is a single nucleotide polymorphism and may be c, t or g
ggccagaagcaattggtccgggaccacacaggcctcgcctcctccgagccctttctttgcttcacttcccctttccgagaacgtccggattcctattggactttggagcgtagagctccgaagctctggattggctggaactcgacgggagatggtggttcggcaaagtgcgcgcgcgcagcaggcggcgggaaggggcgggccaggccgagagtgctcggctttctctggccccaccccctccgccgcgntcttgtcgccgtggtgactgctctacgattggcgggctttggggttcaaaggcatcagcgccgcctcatccgggccctccgactgcggtctctccgggctgattggctagtttcttgcagccctgattggccgagatcggaaaagacggccgggagcttggcgaggtagcaaatgctatggtcgcagggctcaaaatggctccggcctccttcggtgacgtagagggcagaactggggctcgcccctcc

SEQ ID NO: 17 - DNA, 500 bp, Canis familiaris
  misc_feature (251)..(251): n is a single nucleotide polymorphism and may be a or t
cttgggcagggctggattcagtaattttgaggaagcgccaccttcccctgtgagtgacacatctttaagtcttctttttaacctatttgcagattggagagggaagaacagggagggggttattgccaaatatgttaaatgtgggttggggtgcttgtgtatgtatctccctcaatttccccagaaacgaggcattctttttttctcagtctaaaatcaagagggtggggggagagaggaggcatgtcatnaaaagccaggtgtgggggaaagtcaaatcaccagtgtcccatccttggccagaaccctaccatctgagtccctcaaacatcctcaggattttataagactgtcatagtggggaacctctcctgtcaaagaccaggcaggactggaggggagcaggttagatgggtgatgggtggagggtgggaggcacgggccgggggcagttctctcctcacttgtaaacttgtagtttcacagaaaaggaaaaaaaa

SEQ ID NO: 18 - DNA, 500 bp, Canis familiaris
  misc_feature (251)..(251): n is a single nucleotide polymorphism and may be t or g
acgggccgggggcagttctctcctcacttgtaaacttgtagtttcacagaaaaggaaaaaaaaaatgcagttttaaataaagaaatttcttttttccctgggtttagttgagcattattttcaaaaacatgataaaccccagaataaaattctttcataaaaccccaaacggtgttttcccttccagctacccactcccaaccttaccctcaccacccaggagcacccatggttcaccctcaaccctcccncaaggccaggccaggcctgaaccctgttgcccagcaaccttacctaagcaacatggggctcccatcgtccaccaggcaagccctcagtggactgatggaatgggttaggggtcctgaatactaagaaaccttaggaggtccactcccaacccccacggacatggctgtgcccagactggcactgcctaagggtggggtgatcattgtttctcctagtacctgaaggactcttgtctaagaagcatgaat

SEQ ID NO: 19 - DNA, 500 bp, Canis familiaris
  misc_feature (251)..(251): n is a single nucleotide polymorphism and may be c or t
gcggtctctccgggctgattggctagtttcttgcagccctgattggccgagatcggaaaagacggccgggagcttggcgaggtagcaaatgctatggtcgcagggctcaaaatggctccggcctccttcggtgacgtagagggcagaactggggctcgcccctccctttcagtgaagaagggagcagaactggcatcagctcgacgtgtgggtggtgcgagcatcgacctgcggcgctggtgttaacccanctccctcggctgcgggacgcagcccgctcctccttaggtgacttgcagaacagatctcaaggcccacacctttctaacgttgacacaggatgacagagttgaccccggccccgttttaaacctgaaaagcgaactagctccaccccttcgtgagtaggtgccgagggggcaaggcccgccctcctgagcgacccgcggcggaatggggttaggcccgccccttccgtcctgtagtgtgtcccgcagaag

SEQ ID NO: 20 - DNA, 500 bp, Canis familiaris
  misc_feature (251)..(251): n is a single nucleotide polymorphism and may be c or t
ccctcccagggccccatttctcacccctgcccctcgcctgggcttctcttaacagaaggtgtgaatctcatggtcaaattcatgctaagaagtgcgtcatagacctcaacacacatgctttttgaattcttgaaaaataagctggtggagacaacactgcagggagggacccttcctcagggtctgaagatttaaatgaatacaggcaggtaggtccaggtggatggagccgggtctgggccaggcagganagttctggaggaaaacaagagacctcttcagggaatgcccttctccttgggtctcatccccagggttgtgatatcttcttgttcttggcactgagtggatccaaacccgccaggtagggaaacttattttgagactgatcagatttatccatgtcactagatgcttgcctgtagtctcctgtggacccgagggtgctcatccacctcagcttctctttccggcttttttcctccccttcctgccccatc

SEQ ID NO: 21 - DNA, 500 bp, Canis familiaris
  misc_feature (251)..(251): n is a single nucleotide polymorphism and may be c or t
catgcgacagaattggtgtccgttggacctggtcggggagcttgattcgtccgagtagctgccagtcagggccaagggccagaagcaattggtccgggaccacacaggcctcgcctcctccgagccctttctttgcttcacttcccctttccgagaacgtccggattcctattggactttggagcgtagagctccgaagctctggattggctggaactcgacgggagatggtggttcggcaaagtgcgcgngcgcagcaggcggcgggaaggggcgggccaggccgagagtgctcggctttctctggccccaccccctccgccgcggtcttgtcgccgtggtgactgctctacgattggcgggctttggggttcaaaggcatcagcgccgcctcatccgggccctccgactgcggtctctccgggctgattggctagtttcttgcagccctgattggccgagatcggaaaagacggccgggagcttggcgaggtagcaaa

SEQ ID NO: 22 - DNA, 500 bp, Canis familiaris
  misc_feature (251)..(251): n is a single nucleotide polymorphism and may be t or g
agtttcacagaaaaggaaaaaaaaaatgcagttttaaataaagaaatttcttttttccctgggtttagttgagcattattttcaaaaacatgataaaccccagaataaaattctttcataaaaccccaaacggtgttttcccttccagctacccactcccaaccttaccctcaccacccaggagcacccatggttcaccctcaaccctcccccaaggccaggccaggcctgaaccctgttgcccagcaacnttacctaagcaacatggggctcccatcgtccaccaggcaagccctcagtggactgatggaatgggttaggggtcctgaatactaagaaaccttaggaggtccactcccaacccccacggacatggctgtgcccagactggcactgcctaagggtggggtgatcattgtttctcctagtacctgaaggactcttgtctaagaagcatgaattcctagcattccccgtggccggataggacaggatggaaa

SEQ ID NO: 23 - DNA, 500 bp, Canis familiaris
  misc_feature (251)..(251): n is a single nucleotide polymorphism and may be c or t
gccgtgtgttggggggatgcggctagggagagtagaacagggtagcaatcttaagacttcctggccactgcacttccaccttttccaataaggcaccccggagccagggctacaggctcacagaccagcccaggccagtgggtctccgaggggctgagctcacctggctactgtcactgctcagccctggtgatggtgggttgaagctaaagagctcctgtcctctcctgcccgctgtccttccctctgcncctccttctctgccttcccttcaaccaattagtgacttcctccctcccagggccccatttctcacccctgcccctcgcctgggcttctcttaacagaaggtgtgaatctcatggtcaaattcatgctaagaagtgcgtcatagacctcaacacacatgctttttgaattcttgaaaaataagctggtggagacaacactgcagggagggacccttcctcagggtctgaagatttaaatgaatacaggca